(19) United States
(12) Patent Application Publication
(10) Pub. No.: US 2008/0058339 A1
(43) Pub. Date: Mar. 6, 2008

(54) New compounds

(75) Inventors: Peter Brandt, Uppsala (SE); Gary Johansson, Solna (SE); Lars Johansson, Bromma (SE); Tobias Koolmeister, Uppsala (SE); Bjorn Nilsson, Stockholm (SE); Teresa Sandvall, Stockholm (SE)

Correspondence Address: EDWARDS ANGELL PALMER & DODGE LLP, P.O. BOX 55874, BOSTON, MA, 02205, US

(73) Assignee: Biovitrum, Stockholm (SE)

(21) Appl. No.: 11/897,392

(22) Filed: Aug. 29, 2007

Related U.S. Application Data
(60) Provisional application No. 60/860,737, filed on Nov. 21, 2006.

(30) Foreign Application Priority Data
Aug. 30, 2006 (SE) 0601775-0

(51) Int. Cl. A61K 31/501; A61P 25/00; A61P 3/00; A61P 9/00; C07D 401/12

(52) U.S. Cl. 514/252.03; 544/238

(57) ABSTRACT

The present invention relates to compounds of Formula (I) 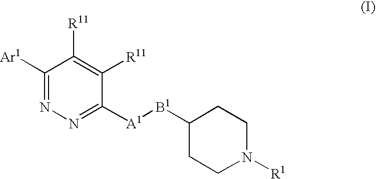 and pharmaceutically acceptable salts, hydrates, geometrical isomers, racemates, tautomers, optical isomers and N-oxides thereof. The invention also relates to pharmaceutical compositions comprising these compounds, and to the use of these compounds for the prophylaxis and treatment of medical conditions relating to disorders of the G-protein-coupled receptor GPR119, such as diabetes and obesity.

### CROSS-REFERENCE TO RELATED APPLICATIONS

This application claims benefit of U.S. provisional application 60/860,737 filed Nov. 21, 2006, and Swedish application 0601775-0 filed Aug. 30, 2006, the entire contents of each which is herein incorporated by reference.

### FIELD OF INVENTION

The present invention relates to certain novel compounds, to pharmaceutical compositions comprising these novel compounds, and to the use of these compounds for the prophylaxis and treatment of medical conditions relating to disorders of the G-protein-coupled receptor GPR119 such as diabetes and obesity.

### BACKGROUND ART

Diabetes mellitus is a group of disorders characterized by abnormal glucose homeostasis resulting in high levels of blood glucose. The most common cases of diabetes mellitus are Type 1 (also referred to as insulin-dependent diabetes mellitus or IDDM) and Type 2 diabetes (also referred to as non-insulin-dependent diabetes mellitus or NIDDM). Type 2 diabetes accounts for approximately 90% of all diabetic cases. Type 2 diabetes is a serious progressive disease that results in the development of microvascular complications (e.g. retinopathy, neuropathy, nephropathy) as well as macrovascular complications (e.g. accelerated atherosclerosis, coronary heart disease, stroke). More than 75% of people with Type 2 diabetes die of cardiovascular diseases.

The increasing prevalence of obesity together with an ageing population is contributing to the predicted explosion in diabetes across the globe. Current projections suggest that 300 million people worldwide have diabetes by 2025.

The pathogenesis of Type 2 diabetes involves insulin resistance, insulin secretory dysfunction (i.e. pancreatic beta cell dysfunction) and hepatic glucose overproduction. Insulin resistance is highly correlated with obesity. Accumulating reports suggest insulin resistance to be central to a cluster of metabolic abnormalities—including dyslipidemia, hypertension, endothelial dysfunction, reduced fibrinolysis, and chronic systemic inflammation—that together are responsible for the increased cardiovascular risk. Current antidiabetic therapy is targeting the defects mentioned above. For instance, sulphonylureas increase production of endogenous insulin. However, this enhanced insulin production is not glucose dependent and there is risk for developing hypoglycaemia. Metformin lowers hepatic glucose output. Thiazolidindiones (TZDs) reduce insulin resistance in muscle and liver and suppress inflammatory responses. A major side effect of TZDs is weight gain due to fluid retention and increase in total body fat. An earlier drug in this class, troglitazone, was withdrawn due to rare but serious cases of hepatotoxicity. Current therapies have limited durability and/or significant side effects.

The widespread availability and increased consumption of Western diet combined with the adoption of a sedentary life-style has increased the number of obese people. Obesity is linked to a wide range of medical complications, such as diabetes, cardiovascular disease and cancer. In addition, being overweight can exacerbate the development of osteoporosis and asthma. Obesity is also proven to double the risk of hypertension. Obesity has only recently been regarded as a disease in the sense of being a specific target for medical therapy. Current therapies for obesity are based on diet and exercise and stomach surgery for extremely obese patients. Two weight loss medications are today available for long-term use. Sibutramine, a serotonin- and noradrenaline-reuptake inhibitor, controls appetite by producing a feeling of satiety. However, a prominent side effect is hypertension. Orlistat inhibits the lipase-mediated breakdown of fat in the gastrointestinal tract, thereby limiting caloric intake resulting in weight loss. However, approximately 20% of the patients using Orlistat develop faecal incontinence and urgency. Thus, there is an unmet medical need for new and novel antidiabetic and antiobesity therapies.

GPR119 (GenBank No. NM 178471) is a G-protein coupled receptor identified as SNORF25 in WO 00/50562. In humans, GPR119 is selectively expressed in pancreas and gastrointestinal tract. Activation of GPR119 by lysophosphatidylcholine (LPC) induces glucose-dependent insulin secretion from pancreatic beta-cells (Soga et al., Biochem. Biophys. Res. Commun. 326, 744-751, 2005). GPR119 agonists stimulate insulin secretion in rat islets and reduce blood glucose in diabetic Lepr^{db/db} mice (WO 2004/065380). Another endogenous ligand for GPR119, oleoylethanolamide (OEA), and a small molecule GPR119 agonist, PSN632408, both suppress food intake and reduce body weight gain in rat (Overton et al., Cell Metabolism 3, 167-175, 2006). Taken together, these data suggest that GPR119 is an interesting target for treating diabetes and/or obesity.

WO 2004/065380, WO 2004/076413, WO 2005/007647, WO 2005/007658 and WO 2005/121121 discloses compounds that are modulators of the Rup3 receptor, also referred to as SNORF25 (WO 00/50562) or as GPR119 (Fredriksson et al., FEBS Lett, 554, 381-388), and which inter alia may be used for the treatment of metabolic disorders and complications thereof, such as, diabetes and obesity.

WO 2005/061489, WO 2006/067531, WO 2006/067532 and WO 2006/070208 disclose compounds that are agonists of GPR116, also referred to as SNORF25 or as GPR119 (see Overton et al, Cell Metabolism 3, 167-175, 2006), and which inter alia may be used for the treatment of metabolic disorders and complications thereof, such as diabetes and obesity.

WO 2006/076231 discloses a synergistic effect of a GPR119 agonist in combination with a DPP-IV inhibitor, in lowering elevated glucose levels in mice. Further, a synergistic effect with the said combination is shown in increasing blood GLP-1 levels after glucose challenge in mice.

### DISCLOSURE OF THE INVENTION

It has surprisingly been found that compounds of the Formula (I) are active as agonists of GPR119 and are potentially useful in the treatment or prophylaxis of disorders relating to GPR119. Examples of such disorders include Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. For parts of the range “C₁₋₆-alkyl”, all subgroups thereof are contemplated, such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. Examples of said C₁₋₆-alkyl include methyl, ethyl, n-propyl, isopropyl, n-butyl, isobutyl, sec-butyl, t-butyl and straight- and branched-chain pentyl and hexyl.

Unless otherwise stated or indicated, the term “cyano-C₁₋₆-alkyl” denotes a C₁₋₆-alkyl group, as defined above, substituted with a cyano group. Exemplary cyano-C₁₋₆-alkyl groups include 2-cyanoethyl and 3-cyanopropyl.

Unless otherwise stated or indicated, the term “amino-C₁₋₆-alkyl” denotes a C₁₋₆-alkyl group, as defined above, substituted with an amino group. Exemplary amino-C₁₋₆-alkyl groups include 2-aminoethyl and 3-aminopropyl.

Unless otherwise stated or indicated, the term “hydroxy-C₁₋₆-alkyl” denotes a straight or branched alkyl group that has a hydrogen atom thereof replaced with OH. Examples of said hydroxy-C₁₋₆-alkyl include hydroxymethyl, 2-hydroxyethyl, 2-hydroxypropyl, 3-hydroxy-3-methylbutyl, 2-hydroxybutyl and 2-hydroxy-2-methylpropyl.

Derived expressions such as “C₁₋₆-alkoxy”, “C₁₋₆-alkylthio” and “C₁₋₆-alkylamino” are to be construed accordingly where an C₁₋₆-alkyl group is attached to the remainder of the molecule through an oxygen, sulfur or nitrogen atom, respectively. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc. Examples of said “C₁₋₆-alkoxy” include methoxy, ethoxy, n-propoxy, isopropoxy, n-butoxy, isobutoxy, sec-butoxy, t-butoxy and straight- and branched-chain pentoxy and hexoxy etc. Subgroups of “C₁₋₆-alkylthio” and “C₁₋₆-alkylamino” are to be construed accordingly.

Unless otherwise stated or indicated, the term “C₁₋₄-alkylsulfinyl” denotes a group C₁₋₄-alkyl-S(O)—. Exemplary C₁₋₄-alkylsulfinyl groups include methylsulfinyl and ethylsulfinyl. Unless otherwise stated or indicated, the term “dihydroxy-C₂₋₆-alkyl” denotes a C₂₋₆-alkyl group which is disubstituted with hydroxy and wherein said hydroxy groups are attached to different carbon atoms. Exemplary dihydroxy-C₂₋₆-alkyl groups include 2,3-dihydroxy-propyl and 2,4-dihydroxybutyl.

Unless otherwise stated or indicated, the term “di(C₁₋₄-alkyl)amino” denotes a group (C₁₋₄-alkyl)₂N—, wherein the two alkyl portions may be the same or different. Exemplary di(C₁₋₄-alkyl)amino groups include N,N-dimethylamino, N-ethyl-N-methylamino and N,N-diethylamino.

Unless otherwise stated or indicated, the term “di(C₁₋₄-alkyl)amino-C₂₋₄-alkyl” denotes a group di(C₁₋₄-alkyl)amino, as defined above, attached to a C₂₋₄-alkyl group. Exemplary di(C₁₋₄-alkyl)amino-C₂₋₄-alkyl groups include 2-(dimethylamino)ethyl and 3-(diethyl-amino)propyl.

Unless otherwise stated or indicated, the term “fluoro-C₁₋₆-alkyl” denotes a C₁₋₆-alkyl group substituted by one or more fluorine atoms. Examples of said fluoro-C₁₋₆-alkyl include 2-fluoroethyl, fluoromethyl, 2-fluoro-1-(fluoromethyl)ethyl, trifluoromethyl, 3,3,3-trifluoropropyl and 2,2,2-trifluoroethyl. Likewise, “aryl-C₁₋₆-alkyl” means a C₁₋₆-alkyl group substituted by an aryl group. Examples include benzyl, 2-phenylethyl, 1-phenylethyl and 2-methyl-2-phenylpropyl.

Unless otherwise stated or indicated, the term “arylcarbonyl-C₁₋₄-alkyl” denotes an arylcarbonyl group (e.g., benzoyl) that is attached through a C₁₋₄-alkyl group. Examples of said arylcarbonyl-C₁₋₄-alkyl include 3-oxo-3-phenylpropyl, 2-oxo-2-phenylethyl and 1-methyl-3-oxo-3-phenylpropyl.

Unless otherwise stated or indicated, the term “heteroarylcarbonyl-C₁₋₄-alkyl” denotes a heteroarylcarbonyl group (e.g., 3-pyridinylcarbonyl) that is attached through a C₁₋₄-alkyl group. Examples of said heteroarylcarbonyl-C₁₋₄-alkyl include 3-oxo-3-(3-pyridinyl)-propyl, 2-oxo-2-(3-pyridinyl)ethyl and 1-methyl-3-oxo-3-(3-pyridinyl)propyl.

Unless otherwise stated or indicated, the term “C₁₋₆-alkoxy-C₂₋₆-alkyl” denotes a straight or branched alkoxy group having from 1 to 6 carbon atoms connected to an alkyl group having from 2 to 6 carbon atoms. Examples of said C₁₋₆-alkoxy-C₂₋₆-alkyl include methoxyethyl, ethoxyethyl, isopropoxyethyl, n-butoxyethyl, t-butoxyethyl and straight- and branched-chain pentoxyethyl. For parts of the range “C₁₋₆-alkoxy-C₂₋₆-alkyl” all subgroups thereof are contemplated such as C₁₋₅-alkoxy-C₂₋₆-alkyl, C₁₋₄-alkoxy-C₂₋₆-alkyl, C₁₋₃-alkoxy-C₂₋₆-alkyl, C₁₋₂-alkoxy-C₂₋₆-alkyl, C₂₋₆-alkoxy-C₂₋₆-alkyl, C₂₋₅-alkoxy-C₂₋₆-alkyl, C₂₋₄-alkoxy-C₂₋₆-alkyl, C₂₋₃-alkoxy-C₂₋₆-alkyl, C₃₋₆-alkoxy-C₂₋₆-alkyl, C₄₋₅-alkoxy-C₂₋₆-alkyl, C₁₋₆-alkoxy-C₂₋₅-alkyl, C₁₋₆-alkoxy-C₂₋₄-alkyl, etc.

Unless otherwise stated or indicated, the term “C₂₋₆-alkenyl” denotes a straight or branched hydrocarbon chain radical containing one carbon-carbon double bond and having from 2 to 6 carbon atoms. Examples of said C₂₋₆-alkenyl include vinyl, allyl, 2,3-dimethylallyl, 1-butenyl, 1-pentenyl, and 1-hexenyl. For parts of the range “C₂₋₆-alkenyl”, all subgroups thereof are contemplated such as C₂₋₅-alkenyl, C₂₋₄-alkenyl, C₂₋₃-alkenyl, C₃₋₆-alkenyl, C₄₋₅-alkenyl, etc. Likewise, “aryl-C₂₋₆-alkenyl” means a C₂₋₆-alkenyl group substituted by an aryl group. Examples of said aryl-C₂₋₆-alkenyl include styryl and cinnamyl.

Unless otherwise stated or indicated, the term “C₂₋₆-alkynyl” denotes a straight or branched hydrocarbon chain radical containing one carbon-carbon triple bond and having from 2 to 6 carbon atoms. Examples of said C₂₋₆-alkynyl include ethynyl, 1-propynyl, 2-propynyl, 1-butynyl, 2-butynyl, and 1-methylprop-2-yn-1-yl.

Likewise, aryl-C₂₋₆-alkynyl means a C₂₋₆-alkynyl group substituted by an aryl group. Examples of said aryl-C₂₋₆-alkynyl include phenylethynyl, 3-phenyl-1-propyn-1-yl, 3-phenyl-2-propyn-1-yl and 4-phenyl-2-butyn-1-yl. The term “oxo” denotes

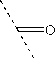

Unless otherwise stated or indicated, the term “C₃₋₇-cycloalkyl” denotes a cyclic alkyl group having a ring size from 3 to 7 carbon atoms and includes cyclopropyl, cyclobutyl, cyclopentyl, cyclohexyl, and cycloheptyl. For parts of the range “C₃₋₇-cycloalkyl” all subgroups thereof are contemplated such as C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₆₋₇-cycloalkyl.

Unless otherwise stated or indicated, the term “C₃₋₇-cycloalkyl-C₁₋₄-alkyl” denotes a C₃₋₇-cycloalkyl group attached to a C₁₋₄-alkyl group. Exemplary C₃₋₇-cycloalkyl-C₁₋₄-alkyl groups include cyclopropylmethyl, 1-cyclopropylethyl, cyclohexylmethyl and 2-cyclo-hexylethyl. When the cycloalkyl portion as part of the group C₃₋₇-cycloalkyl-C₁₋₄-alkyl is substituted with methyl, examples of such groups include (1-methylcyclopropyl)methyl and 2-(4-methylcyclohexyl)ethyl.

Unless otherwise stated or indicated, the term “C₇₋₈-bicyclyl” denotes a carbobicyclic saturated aliphatic ring system in which two non-adjacent carbon atoms of a monocyclic ring are linked by an alkylene bridge of between one and three additional carbon atoms. Examples of said C₇₋₈-bicyclyl include radicals obtainable from bicyclo[3.1.1]heptane, bicyclo[2.2.1]heptane (norbornane) and bicyclo[2.2.2]octane.

Unless otherwise stated or indicated, the term C₇₋₈-bicyclylalkyl means a C₁₋₆-alkyl group substituted by a C₇₋₈-bicyclyl group as defined above. An exemplary C₇₋₈-bicyclylalkyl group is bicyclo[2.2.1]hept-2-ylmethyl (2-norbonylmethyl).

Unless otherwise stated or indicated, the term “C₅₋₈-cycloalkenyl” denotes a monocyclic or bicyclic alkenyl group of 5 to 8 carbon atoms having one carbon-carbon double bond. Examples of monocyclic cycloalkenyl groups are cyclopent-3-en-1-yl and cyclohexen-1-yl. An exemplary bicyclic cycloalkenyl group is bicyclo[2.2.1]hept-5-en-2-yl (norbornen-2-yl).

Unless otherwise stated or indicated, the term “oxo-C₄₋₆-cycloalkyl” refers to a C₄₋₆-cycloalkyl wherein one of the ring carbons is a carbonyl. Examples of “oxo-C₄₋₆-cycloalkyl” include 2-oxocyclobutyl, 3-oxocyclobutyl, 2-oxocyclopentyl and 4-oxo-cyclohexyl.

Unless otherwise stated or indicated, the term “fluoro-C₃₋₆-cycloalkyl” denotes a C₃₋₆-cycloalkyl group substituted by one or two fluorine atoms. Examples of said “fluoro-C₃₋₆-cycloalkyl” include 2,2-difluorocyclopropyl and 4-fluorocyclohexyl.

Unless otherwise stated or indicated, the term “C₁₋₃-alkoxy-C₄₋₆-cycloalkyl” denotes a C₄₋₆-cycloalkyl group substituted by a C₁₋₃-alkoxy group. Examples of said “C₁₋₃-alkoxy-C₄₋₆-cycloalkyl” include 4-methoxycyclohexyl and 2-ethoxycyclopentyl.

Unless otherwise stated or indicated, the term “methyl-C₃₋₆-cycloalkyl” denotes a C₃₋₆-cycloalkyl group substituted by one or two methyl groups. Examples of said “methyl-C₃₋₆-cycloalkyl” include 4-methylcyclohexyl and 3,3-dimethylcyclopentyl.

Unless otherwise stated or indicated, the term “acyl”, which may be straight or branched, denotes a carbonyl group that is attached through its carbon atom to a hydrogen atom to form a C₁-acyl group (i.e., a formyl group) or to an alkyl group, where alkyl is defined as above. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc. Exemplary acyl groups include formyl, acetyl (i.e., C₂-acyl), propanoyl, butanoyl, pentanoyl, hexanoyl.

Unless otherwise stated or indicated, the term “C₂₋₆-acyl-C₁₋₆-alkyl” refers to a group C₁₋₅-alkyl-(C═O)—C₁₋₆-alkyl. Exemplary C₂₋₆-acyl-C₁₋₆-alkyl groups include 2-acetylethyl and 3-acetylpropyl.

Unless otherwise stated or indicated, the term “C₁₋₆-alkylsulfonyl”, which may be straight or branched, denotes a hydrocarbon having from 1 to 6 carbon atoms with a sulfonyl group. For parts of the range “C₁₋₆-alkylsulfonyl” all subgroups thereof are contemplated such as C₁₋₅-alkylsulfonyl, C₁₋₄-alkylsulfonyl, C₁₋₃-alkylsulfonyl, C₁₋₂-alkylsulfonyl, C₂₋₆-alkylsulfonyl, C₂₋₅-alkylsulfonyl, C₂₋₄-alkylsulfonyl, C₂₋₃-alkylsulfonyl, C₃₋₆-alkylsulfonyl, C₄₋₅-alkylsulfonyl, etc. Exemplary C₁₋₆-alkylsulfonyl groups include methylsulfonyl, ethylsulfonyl, propylsulfonyl, n-butylsulfonyl, sec-butylsulfonyl, tert-butylsulfonyl, pentylsulfonyl and hexylsulfonyl.

Unless otherwise stated or indicated, the term “hydroxy-C₂₋₄-alkylsulfonyl” denotes a C₂₋₄-alkylsulfonyl group as defined above substituted with a hydroxy group. Examples of said hydroxy-C₂₋₄-alkylsulfonyl include hydroxymethylsulfonyl and 2-hydroxyethylsulfonyl.

Unless otherwise stated or indicated, the term “C₁₋₄-alkylsulfonamido” denotes a group C₁₋₄-alkyl-SO₂NH—. Exemplary C₁₋₄-alkylsulfonamido groups include methylsulfonyl-amino and ethylsulfonylamino.

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring, preferably mono- or bicyclic. Examples of aryls are phenyl, indenyl, 2,3-dihydroindenyl (indanyl), 1-naphthyl, 2-naphthyl or 1,2,3,4-tetra-hydronaphthyl.

Unless otherwise stated or indicated, the term “heteroaryl” refers to a mono- or bicyclic heteroaromatic ring system having 5 to 10 ring atoms in which one or more of the ring atoms are other than carbon, such as nitrogen, sulphur or oxygen. Only one ring need be aromatic and said heteroaryl moiety can be linked to the remainder of the molecule via a carbon or nitrogen atom in any ring. Examples of heteroaryl groups include furyl, pyrrolyl, thienyl, oxazolyl, isoxazolyl, imidazolyl, thiazolyl, isothiazolyl, pyridyl, pyrimidinyl, quinazolinyl, indolyl, isoindolyl, 1,3-dihydro-isoindolyl, pyrazolyl, pyridazinyl, quinolinyl, quinoxalinyl, thiadiazolyl, benzofuranyl, 2,3-dihydrobenzofuranyl, 1,3-benzodioxolyl, 1,4-benzodioxinyl, 2,3-dihydro-1,4-benzodioxinyl, benzothiazolyl, benzimidazolyl, benzothiadiazolyl, benzotriazolyl, indolinyl, isoindolinyl, and chromanyl groups.

Unless otherwise stated or indicated, the term “heterocyclyl” or “heterocyclic ring” refers to a non-aromatic fully saturated or partially unsaturated monocyclic ring system having 4 to 7 ring atoms with at least one heteroatom such as O, N, or S, and the remaining ring atoms are carbon. Examples of heterocyclic groups include piperidinyl, tetrahydropyranyl, tetrahydrofuranyl, oxetanyl, azepinyl, azetidinyl, pyrrolidinyl, morpholinyl, imidazolinyl, imidazolidinyl, thiomorpholinyl, pyranyl, dioxanyl, piperazinyl and 5,6-dihydro-4H-1,3-oxazin-2-yl. When present, the sulfur atom may be in an oxidized form (i.e., S═O or O═S═O). Exemplary heterocyclic groups containing sulfur in oxidized form are 1,1-dioxido-thiomorpholinyl and 1,1-dioxido-isothiazolidinyl.

When two groups R⁵, two groups R^{5A}, two groups R⁹ or two groups R^{9A} described herein form a heterocyclic ring and said heterocyclic ring is substituted with one or two oxo groups, examples of such groups include 2-pyrrolidon-1-yl, 2-piperidon-1-yl, 2-azetidinon-1-yl, 2,5-dioxopyrrolidin-1-yl and hydantoin-1-yl (i.e., 2,5-dioxoimidazolidin-1-yl). When two groups R⁵, two groups R^{5A}, two groups R⁹ or two groups R^{9A} described herein form a heterocyclic ring and said heterocyclic ring is substituted with one or two fluoro atoms, examples of such groups include 4-fluoropiperidin-1-yl, 4,4-difluoropiperidin-1-yl, 3-fluoropyrrolidin-1-yl and 3,3-difluoropyrrolin-1-yl.

When two groups R⁵, two groups R^{5A}, two groups R⁹ or two groups R^{9A} described herein form a heterocyclic ring and said heterocyclic ring is substituted with hydroxy, examples of such groups include 4-hydroxypiperidin-1-yl, 3-hydroxypiperidin-1-yl, and 3-hydroxy-pyrrolidin-1-yl.

When two groups R⁵, two groups R^{5A}, two groups R⁹ or two groups R^{9A} described herein form a heterocyclic ring and said heterocyclic ring is substituted with amino, examples of such groups include 4-aminopiperidin-1-yl, 3-aminopiperidin-1-yl, and 3-aminopyrrolidin-1-yl.

When two groups R⁵, two groups R^{5A}, two groups R⁹ or two groups R^{9A} groups described herein form a heterocyclic ring and said heterocyclic ring is substituted with hydroxymethyl, examples of such groups include 2-(hydroxymethyl)pyrrolidin-1-yl, 2-(hydroxymethyl)morpholin-4-yl and 4-(hydroxymethyl)piperidin-1-yl.

When two groups R⁵, two groups R^{5A}, two groups R⁹ or two groups R^{9A} groups described herein form a heterocyclic ring and said heterocyclic ring is substituted with methylamino or dimethylamino, examples of such groups include 3-dimethylaminopyrrolidin-1-yl and 3-methylaminopyrrolidin-1-yl.

Unless otherwise stated or indicated, the term “heteroaryl-C₁₋₄-alkyl” denotes a heteroaryl group that is attached through a C₁₋₄-alkyl group. Examples of said heteroaryl-C₁₋₄-alkyl include 2-(pyridin-2-yl)ethyl and 1,3 benzodioxol-5-ylmethyl.

“C-heterocyclyl” indicates bonding via a carbon atom of said heterocyclyl, for example piperidin-4-yl, tetrahydrofuran-2-yl, oxetan-3-yl, tetrahydrofuran-3-yl and 5,6-dihydro-4H-1,3-oxazin-2-yl, while “N-heterocyclyl” indicates bonding through nitrogen in a nitrogen-containing heterocyclyl group, for example piperidin-1-yl and piperazin-1-yl. When C-heterocyclyl is substituted by C₁₋₄-alkyl, said C₁₋₄-alkyl is attached to a ring nitrogen atom or a ring carbon atom thereof. Exemplary C-heterocyclyl groups substituted by C₁₋₄-alkyl include 1-methylpiperidin-4-yl and 3-methyloxetan-3-yl.

Unless otherwise stated or indicated, the term “N-heterocyclyl-C₂₋₄-alkyl” refers to a nitrogen-containing heterocyclyl group that is directly linked to a C₂₋₄-alkyl group via a nitrogen atom of said heterocyclyl. Exemplary N-heterocyclyl-C₂₋₄-alkyl groups include 2-(pyrrolidin-1-yl)ethyl, 3-(4-morpholinyl)propyl, 2-(piperazin-1-yl)ethyl and 2-(4-morpholinyl)ethyl.

When heterocyclyl as part of the group N-heterocyclyl-C₂₋₄-alkyl is substituted by methyl, said heterocyclyl is selected from 1-piperazinyl or 1-homopiperazinyl and said methyl is attached to the 4-position of the piperazine or homopiperazine ring. Exemplary N-heterocyclyl-C₂₋₄-alkyl groups wherein heterocyclyl is substituted with methyl are 2-(4-methylpiperazin-1-yl)ethyl, 2-(4-methylhomopiperazin-1-yl)ethyl.

Unless otherwise stated or indicated, the term “C-heterocyclyl-C₁₋₄-alkyl” refers to a heterocyclyl group that is directly linked to a C₁₋₄-alkyl group via a carbon atom of said heterocyclyl. Exemplary C-heterocyclyl-C₁₋₄-alkyl groups include tetrahydropyran-4-yl-methyl, piperidin-4-ylmethyl, tetrahydrofuran-2-ylmethyl, oxetan-3-ylmethyl and 2-(piperidinyl-4-yl)ethyl.

When heterocyclyl as part of the group C-heterocyclyl-C₁₋₄-alkyl is substituted by methyl, said methyl is attached to a ring nitrogen atom or ring carbon atom thereof. Exemplary C-heterocyclyl-C₁₋₄-alkyl groups wherein heterocyclyl is substituted with methyl are 2-(1-methylpiperidin-4-yl)ethyl and 3-methyloxetan-3-ylmethyl.

Unless otherwise stated or indicated, the term “oxo-N-heterocyclyl” denotes a nitrogen-containing heterocyclyl group that is substituted with one or two oxo groups.

Unless otherwise stated or indicated, the term “oxo-N-heterocyclyl-C₂₋₄-alkyl” refers to an oxo-N-heterocyclyl group that is directly linked to a C₂₋₄-alkyl group through a nitrogen atom of its heterocyclyl portion and where oxo-N-heterocyclyl is as defined above. Exemplary oxo-N-heterocyclyl-C₂₋₄-alkyl groups include 2-(2-pyrrolidon-1-yl)ethyl, 3-(2-pyrrolidon-1-yl)propyl and 2-(2,5-dioxoimidazolidin-1-yl)ethyl.

Unless otherwise stated or indicated, the term “fluoro-N-heterocyclyl” denotes a nitrogen-containing heterocyclyl group that is substituted at a position other than alpha to a ring heteroatom with one or two fluorine atoms.

Unless otherwise stated or indicated, the term “fluoro-N-heterocyclyl-C₂₋₄-alkyl” refers to a fluoro-N-heterocyclyl group that is directly linked to a C₂₋₄-alkyl group through a nitrogen atom of its heterocyclyl portion and where fluoro-N-heterocyclyl is as defined above. Exemplary fluoro-N-heterocyclyl-C₂₋₄-alkyl groups include 2-(3-fluoropyrrolidin-1-yl)-ethyl and 3-(3-fluoropyrrolidin-1-yl)propyl.

Unless otherwise stated or indicated, the term “hydroxy-N-heterocyclyl” denotes a nitrogen-containing heterocyclyl group that is substituted at a position other than alpha to a ring heteroatom with a hydroxy group.

Unless otherwise stated or indicated, the term “hydroxy-N-heterocyclyl-C₂₋₄-alkyl” refers to a hydroxy-N-heterocyclyl group that is directly linked to a C₂₋₄-alkyl group through a nitrogen atom of its heterocyclyl portion and where hydroxy-N-heterocyclyl is as defined above. Exemplary hydroxy-N-heterocyclyl-C₂₋₄-alkyl groups include 2-(4-hydroxy-piperidin-1-yl)ethyl and 3-(3-hydroxypiperidin-1-yl)propyl.

Unless otherwise stated or indicated, the term “amino-N-heterocyclyl” denotes a nitrogen-containing heterocyclyl group that is substituted at a position other than alpha to a ring heteroatom with an amino group.

Unless otherwise stated or indicated, the term “amino-N-heterocyclyl-C₂₋₄-alkyl” refers to a amino-N-heterocyclyl group that is directly linked to a C₂₋₄-alkyl group through a nitrogen atom of its heterocyclyl portion and where amino-N-heterocyclyl is as defined above. Exemplary amino-N-heterocyclyl-C₂₋₄-alkyl groups include 2-(4-aminopiperidin-1-yl)ethyl and 3-(3-aminopiperidin-1-yl)propyl.

Unless otherwise stated or indicated, the term “azabicyclyl” denotes a bicyclic heterocyclyl group with seven or eight atoms (including bridgehead atoms), wherein at least one ring member is a nitrogen atom and the remainder ring atoms being carbon. The said azabicyclyl may optionally contain a carbon-carbon double bond. Examples of azabicyclyl groups include carbon radicals obtainable from 1-azabicyclo[2.2.2]octane, 1-aza-bicyclo[2.2.1]heptane and azabicyclo[2.2.2]oct-2-ene.

“C-heterocyclylsulfonyl” refers to a heterocyclyl group that is directly bonded to SO₂ via a carbon atom. Exemplary C-heterocyclylsulfonyl groups include 4-piperidinylsulfonyl and tetrahydropyran-4-ylsulfonyl.

When C-heterocyclylsulfonyl is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclylsulfonyl group substituted by C₁₋₄-alkyl includes 1-methylpiperidin-4-ylsulfonyl.

Unless otherwise stated or indicated, the term “C₂₋₄-acylamino” denotes a group R^{b}(C═O)NH— wherein R^{b} is selected from C₁₋₃-alkyl. Exemplary C₂₋₄-acylamino groups include acetylamino and propionylamino.

Unless otherwise stated or indicated, the term “C₂₋₄-acylamino-C₁₋₄-alkyl” denotes a C₂₋₄ acylamino group, as defined above, attached to a C₁₋₄-alkyl group. Exemplary C₂₋₄-acylamino-C₁₋₄-alkyl groups include (acetylamino)methyl and 2-(acetylamino)ethyl.

Unless otherwise stated or indicated, the term “aminocarbonyl” refers to the radical NH₂(C═O)—.

Unless otherwise stated or indicated, the term “aminocarbonyl-C₁₋₄-alkyl” denotes a C₁₋₄-alkyl group, as defined above, substituted with an aminocarbonyl group. Exemplary aminocarbonyl-C₁₋₄-alkyl groups include 2-(aminocarbonyl)ethyl and 3-(aminocarbonyl)-propyl.

Unless otherwise stated or indicated, the term “carboxy” denotes a group —C(O)OH.

Unless otherwise stated or indicated, the term “carboxy-C₁₋₃-alkyl” refers to a carboxy group, as defined above, attached to a C₁₋₃-alkyl group. Exemplary carboxy-C₁₋₃-alkyl groups include 2-carboxyethyl and 3-carboxypropyl.

Unless otherwise stated or indicated, the term “carboxy-C₁₋₃-alkylcarbonylamino” refers to a carboxy-C₁₋₃-alkyl groups, as defined above, attached to the carbonyl carbon of carbonylamino (i.e., —C(O)NH—). Exemplary carboxy-C₁₋₃-alkylcarbonylamino groups include (2-carboxyethyl)carbonylamino and (3-carboxypropyl)carbonylamino.

“C-heterocyclylcarbonyl” refers to a heterocyclyl group that is directly bonded to a carbonyl group via a carbon atom while “N-heterocyclylcarbonyl” refers to a nitrogen-containing heterocyclyl group that is directly bonded to a carbonyl group via a nitrogen atom. Examples of N-heterocyclylcarbonyl groups include 1-piperidinylcarbonyl, 1-piperazinylcarbonyl and 1-pyrrolidincarbonyl. Exemplary C-heterocyclylcarbonyl groups include 3-piperidinylcarbonyl, 4-piperidinylcarbonyl and tetrahydropyranyl-4-yl-carbonyl.

When C-heterocyclylcarbonyl is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclylcarbonyl group substituted by C₁₋₄-alkyl includes 1-methylpiperidin-4-ylcarbonyl.

The term “N-heterocyclylcarbonyl-C₂₋₄-alkyl” refers to a N-heterocyclylcarbonyl group that is directly linked to a C₂₋₄-alkyl group through its carbonyl carbon atom and where N-heterocyclylcarbonyl is as defined above. Exemplary N-heterocyclylcarbonyl-C₂₋₄-alkyl groups include 2-(pyrrolidin-1-ylcarbonyl)ethyl, 2-(piperazin-1-ylcarbonyl)ethyl and 2-(piperidin-1-ylcarbonyl)ethyl.

When heterocyclyl as part of the group N-heterocyclylcarbonyl-C₂₋₄-alkyl is substituted by methyl, said heterocyclyl is selected from 1-piperazinyl or 1-homopiperazinyl and said methyl is attached to the 4-position of the piperazine or homopiperazine ring. Exemplary N-heterocyclylcarbonyl-C₂₋₄-alkyl groups wherein heterocyclyl is substituted with methyl are 2-(4-methylpiperazin-1-ylcarbonyl)ethyl, 2-(4-methylhomopiperazin-1-ylcarbonyl)-ethyl.

The term “C-heterocyclylcarbonyl-C₂₋₄-alkyl” refers to a C-heterocyclylcarbonyl group that is directly linked to a C₂₋₄-alkyl group through its carbonyl carbon atom and where C-heterocyclylcarbonyl is as defined above. Exemplary C-heterocyclylcarbonyl-C₂₋₄-alkyl groups include 2-(tetrahydropyran-4-ylcarbonyl)ethyl, 2-(piperidin-3-ylcarbonyl)ethyl and 2-(piperidin-4-ylcarbonyl)ethyl.

When heterocyclyl as part of the group C-heterocyclylcarbonyl-C₂₋₄-alkyl is substituted by methyl, said heterocyclyl is selected from a nitrogen-containing heterocyclyl and said methyl is attached to a ring nitrogen atom thereof. An exemplary C-heterocyclylcarbonyl-C₂₋₄-alkyl group wherein heterocyclyl is substituted with methyl is 2-(1-methylpiperidin-4-ylcarbonyl)ethyl.

The term “C-heterocyclyloxy” refers to a heterocyclic group that is directly bonded to an oxygen atom via a carbon atom. Examples of C-heterocyclyloxy groups include 3-piperidinyloxy, 4-piperidinyloxy, 3-tetrahydrofuranyloxy, and 4-tetrahydropyranyloxy. When C-heterocyclyloxy is substituted by C₁₋₄-alkyl, said heterocyclyl is selected from a nitrogen-containing heterocyclyl, and said C₁₋₄-alkyl is attached to a ring nitrogen atom thereof. An exemplary C-heterocyclyloxy group substituted by C₁₋₄-alkyl includes 1-methylpiperidin-4-yloxy.

The term “hydroxy-C₂₋₄-alkoxy-C₁₋₄-alkyl” refers to a hydroxy-C₂₋₄-alkoxy group that is directly attached to a C₁₋₄-alkyl group. Representative examples of such groups include:

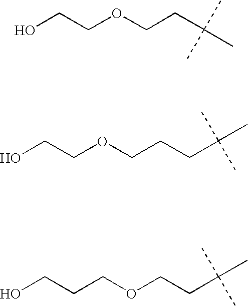

The term “phosphonooxy” refers to a group with the following chemical structure:

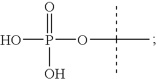

The term “amidino” refers to a group with the following chemical structure:

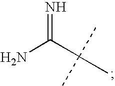

The term “guanidino” refers to a group with the following chemical structure:

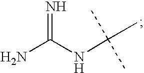

The chemical formula —C(OH)CH₃CF₃ refers to a group with the following chemical structure:

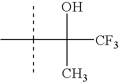

The term [C(OH)CH₃CF₃]-C₁₋₆-alkyl refers to a —C(OH)CH₃CF₃ group that is directly attached to a C₁₋₆-alkyl group. Representative examples of such groups include:

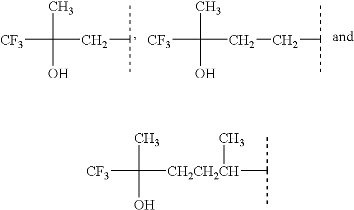

The chemical formula CF₃SO₃ refers to a group with the following chemical structure:

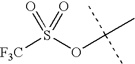

The carbon-carbon double or triple bonds present in the groups C₃₋₆-alkenyl, C₃₋₆-alkynyl, aryl-C₃₋₆-alkenyl and aryl-C₃₋₆-alkynyl as values for R² are meant to be located at positions other than conjugated with a carbonyl group or adjacent to a nitrogen, oxygen or sulfur atom.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “Syndrome X” refers to a syndrome comprising of some or all of the following diseases: 1) dyslipoproteinemia (combined hypercholesterolemia-hypertriglyceridemia, low HDL-cholesterol), 2) obesity (in particular upper body obesity), 3) impaired glucose tolerance (IGT) leading to noninsulin-dependent diabetes mellitus (NIDDM), 4) essential hypertension and (5) thrombogenic/fibrinolytic defects.

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8th ed., Mc-Graw-Hill, Int. Ed. 1992, “Biotransformation of Drugs”, p. 13

The following abbreviations have been used:
BOC means tert-butyloxycarbonyl,
Brine means water saturated or nearly saturated with sodium chloride,
DCM means dichloromethane,
DME means 1,2-dimethoxyethane,
DMF means dimethylformamide,
DMSO means dimethyl sulphoxide,
EDTA means ethylenediamine tetraacetic acid,
ESI means electrospray ionization,
EtOH means ethanol,
EtOAc means ethyl acetate,
HDL means High-Density Lipoprotein,
HPLC means High Performance Liquid Chromatography,
HRESIMS means High-Resolution Electrospray Ionization Mass Spectra,
LCMS means Liquid Chromatography Mass Spectrometry,
LRESIMS means Low-Resolution Electrospray Ionization Mass Spectra,
MeCN means acetonitrile,
MeOH means methanol,
r.t. means room temperature,
R_{T} means retention time,
R_{TA} means retention time system A,
R_{TB} means retention time system B,
t-BuOK means potassium tert-butoxide,
TEA means triethylamine,
TFA means trifluoroacetic acid,
THF means tetrahydrofuran.

The term “leaving group” refers to a group to be displaced from a molecule during a nucleophilic displacement reaction. Examples of leaving groups are iodide, bromide, chloride, methanesulfonyloxy, hydroxy, methoxy, thiomethoxy, toluenesulfonyloxy (tosyl) and trifluoromethanesulfonyloxy (triflate), or suitable protonated forms thereof (e.g., H₂O, MeOH).

The terms “exo” and “endo” are stereochemical prefixes that describe the relative configuration of a substituent on a bridge (not a bridgehead) of a bicyclic system such as 1-azabicyclo[2.2.1]heptane and bicyclo[2.2.1]heptane. If a substituent is oriented toward the larger of the other bridges, it is endo. If a substituent is oriented toward the smaller bridge it is exo. Both exo and endo forms and their mixtures are part of the present invention.

In a first aspect, the present invention provides a compound of Formula (I), 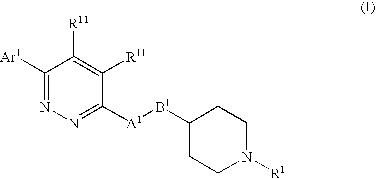
and pharmaceutically acceptable salts, hydrates, geometrical isomers, racemates, tautomers, optical isomers, and N-oxides thereof; wherein:
A¹ is CH₂, O or NR¹⁰;
B¹ is CH₂, O or NR¹⁰, provided that when B¹ is O or NR¹⁰, then A¹ is CH₂;
R¹ is C(O)OR², C(O)R², S(O)₂R², C(O)NR²R³ or —CH₂—C(O)NR²R³;
Ar¹ is phenyl, which is optionally substituted in one or two positions with a substituent independently selected from the group Z¹ consisting of:
(a) CF₃SO₃,
(b) halogen selected from bromine, chlorine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —NR⁶C(O)R⁴,
(h) —CH₂—NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) —NHC(O)OCH₃,
(p) C-heterocyclyl, optionally substituted with methyl,
(q) —CN,
(r) —OR⁸,
(s) —SCF₃,
(t) —NO₂,
(u) phosphonooxy,
(v) C-heterocyclylsulfonyl, optionally substituted with methyl,
(w) —NR⁵R⁵,
(x) —C(OH)CH₃CF₃,
(y) cyano-C₁₋₆-alkyl,
(z) guanidino,
(aa) amidino,
(bb) C₁₋₆-alkyl,
(cc) C₁₋₄-alkoxy-C₁₋₄-alkyl,
(dd) fluoro-C₁₋₄-alkyl,
(ee) C₂₋₆-alkenyl,
(ff) fluoro-C₂₋₄-alkenyl,
(gg) hydroxy-C₁₋₆-alkyl,
(hh) C₁₋₄-alkylsulfonyl-C₁₋₄-alkyl,
(ii) hydroxy-C₂₋₄-alkoxy-C₁₋₄-alkyl,
(jj) C₂₋₃-acyl-C₁₋₃-alkyl,
(kk) C₂₋₆-alkynyl,
(ll) C₃₋₆-cycloalkyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with methyl,
(qq) C₃₋₆-cycloalkyl-C₁₋₄-alkyl,
(rr) R⁵R⁵N—C₁₋₂-alkyl,
(ss) —C(O)OR⁷,
(tt) aryl, and
(uu) heteroaryl,
   wherein any aryl or heteroaryl residue as substituent on Ar¹ is optionally independently substituted in one or more positions with a substituent selected from the group Z² consisting of:
(a) halogen selected from chlorine and fluorine,
(b) C₁₋₄-alkyl,
(c) hydroxy,
(d) C₁₋₄-alkoxy,
(e) —OCF₃,
(f) —SCF₃,
(g) —CN,
(h) —C(OH)CH₃CF₃,
(i) hydroxy-C₁₋₄-alkyl,
(j) —CF₃,
(k) —S(O)₂CH₃,
(l) —S(O)₂NH₂,
(m) —S(O)₂NHCH₃,
(n) —S(O)₂N(CH₃)₂,
(o) —N(CH₃)S(O)₂CH₃,
(p) —N(CH₃)C(O)CH₃,
(q) —C(O)NH₂,
(r) —C(O)NHCH₃,
(s) —C(O)N(CH₃)₂,
(t) —C(O)CH₃,
(u) —NH₂,
(v) —NHCH₃,
(w) —N(CH₃)₂,
(x) —NO₂, and
(y) methoxycarbonyl;
   R² is selected from:
(a) C₁₋₆-alkyl,
(b) C₁₋₆-alkoxy-C₂₋₆-alkyl,
(c) hydroxy-C₂₋₆-alkyl,
(d) fluoro-C₂₋₆-alkyl,
(e) C₃₋₆-alkynyl,
(f) C₃₋₆-alkenyl,
(g) C₃₋₇-cycloalkyl,
(h) C₅₋₈-cycloalkenyl,
(i) NR⁹R⁹, provided that R¹ is not selected from C(O)OR², C(O)NR²R³ and —CH₂—C(O)NR²R³,
(j) C-heterocyclyl, optionally substituted with C₁₋₄-alkyl,
(k) C₇₋₈-bicyclyl, optionally substituted with hydroxy,
(l) C₇₋₈-bicyclylmethyl,
(m) azabicyclyl, optionally substituted with hydroxy,
(n) C₃₋₇-cycloalkyl-C₁₋₄-alkyl, wherein cycloalkyl is optionally substituted with methyl,
(o) C₁₋₆-alkylsulfonyl-C₂₋₆-alkyl,
(p) C₂₋₃-acyl-C₁₋₄-alkyl,
(q) arylcarbonyl-C₁₋₄-alkyl,
(r) heteroarylcarbonyl-C₁₋₄-alkyl,
(s) [C(OH)CH₃CF₃]-C₁₋₆-alkyl,
(t) N-heterocyclylcarbonyl-C₂₋₄-alkyl, wherein heterocyclyl is optionally substituted with methyl,
(u) C-heterocyclylcarbonyl-C₂₋₄-alkyl, wherein heterocyclyl is optionally substituted with methyl,
(v) aminocarbonyl-C₂₋₆-alkyl,
(w) C₁₋₃-alkylaminocarbonyl-C₂₋₆-alkyl,
(x) di(C₁₋₃-alkyl)aminocarbonyl-C₂₋₆-alkyl,
(y) hydroxy-C₂₋₄-alkoxy-C₂₋₄-alkyl,
(z) hydroxy-C₄₋₆-cycloalkyl,
(aa) oxo-C₄₋₆-cycloalkyl,
(bb) fluoro-C₄₋₆-cycloalkyl,
(cc) C₁₋₁₃-alkoxy-C₄₋₆-cycloalkyl,
(dd) methyl-C₃₋₆-cycloalkyl,
(ee) oxo-N-heterocyclyl-C₂₋₄-alkyl,
(ff) fluoro-N-heterocyclyl-C₂₋₄-alkyl,
(gg) amino-N-heterocyclyl-C₂₋₄-alkyl,
(hh) hydroxy-N-heterocyclyl-C₂₋₄-alkyl,
(ii) N-heterocyclyl-C₂₋₄-alkyl, wherein heterocyclyl is optionally substituted with methyl,
(jj) C-heterocyclyl-C₁₋₄-alkyl, wherein heterocyclyl is optionally substituted with methyl,
(kk) aryl,
(ll) aryl-C₁₋₄-alkyl,
(mm) aryl-C₃₋₆-alkenyl,
(nn) aryl-C₃₋₆-alkynyl,
(oo) heteroaryl,
(pp) heteroaryl-C₁₋₄-alkyl,
(qq) heteroaryl-C₃₋₆-alkenyl, and
(rr) heteroaryl-C₃₋₆-alkynyl,
   wherein any aryl or heteroaryl residue, alone or as part of another group, is optionally independently substituted in one or more position with a substituent selected from the group Z² as defined above;
   R³ is selected from:
(a) hydrogen,
(b) C₁₋₆-alkyl,
(c) fluoro-C₂₋₆-alkyl,
(d) hydroxy-C₂₋₆-alkyl,
(e) C₁₋₆-alkoxy-C₂₋₆-alkyl,
(f) amino-C₂₋₆-alkyl,
(g) C₁₋₃-alkylamino-C₂₋₆-alkyl,
(h) di(C₁₋₃-alkyl)amino-C₂₋₆-alkyl,
(i) cyano-C₁₋₆-alkyl, and
(j) C₁₋₆-alkylsulfonyl-C₂₋₆-alkyl;
   R⁴ is independently selected from:
(a) C₁₋₆-alkyl,
(b) fluoro-C₁₋₆-alkyl,
(c) hydroxy-C₂₋₆-alkyl,
(d) C₁₋₄-alkoxy-C₂₋₄-alkyl,
(e) C₂₋₄-acyl-C₁₋₄-alkyl,
(f) carboxy-C₁₋₃-alkyl,
(g) C₃₋₆-cycloalkyl,
(h) oxo-C₄₋₆-cycloalkyl,
(i) hydroxy-C₄₋₆-cycloalkyl,
(j) fluoro-C₄₋₆-cycloalkyl,
(k) methyl-C₃₋₆-cycloalkyl,
(l) N-heterocyclylcarbonyl-C₂₋₄-alkyl, wherein heterocyclyl is optionally substituted with methyl,
(m) oxo-N-heterocyclyl-C₂₋₄-alkyl,
(n) fluoro-N-heterocyclyl-C₂₋₄-alkyl,
(o) hydroxy-N-heterocyclyl-C₂₋₄-alkyl,
(p) amino-N-heterocyclyl-C₂₋₄-alkyl,
(q) aminocarbonyl-C₂₋₄-alkyl,
(r) C₁₋₃-alkylaminocarbonyl-C₂₋₄-alkyl,
(s) di(C₁₋₃-alkyl)aminocarbonyl-C₂₋₄-alkyl,
(t) C₂₋₃-acylamino-C₂₋₄-alkyl,
(u) hydroxy-C₂₋₄-alkoxy-C₂₋₄-alkyl,
(v) C-heterocyclylcarbonyl-C₂₋₄-alkyl, wherein heterocyclyl is optionally substituted with methyl,
(w) C₃₋₆-cycloalkyl-C₁₋₂-alkyl,
(x) aryl,
(y) aryl-C₁₋₂-alkyl,
(z) heteroaryl, and
(aa) heteroaryl-C₁₋₂-alkyl,
   wherein any aryl or heteroaryl residue, alone or as part of another group, is optionally independently substituted in one or more positions with a substituent selected from the group Z³ consisting of:
(a) halogen selected from chlorine and fluorine,
(b) C₁₋₄-alkoxy,
(c) hydroxymethyl,
(d) —CN,
(e) —CF₃,
(f) C₁₋₄-alkyl,
(g) —OCF₃, and
(h) —C(O)CH₃;
   R⁵ is each independently selected from:
(a) hydrogen,
(b) C₁₋₆-alkyl,
(c) C₃₋₄-cycloalkyl,
(d) fluoro-C₂₋₄-alkyl,
(e) amino-C₂₋₆-alkyl,
(f) cyano-C₁₋₆-alkyl,
(g) hydroxy-C₂₋₆-alkyl,
(h) dihydroxy-C₂₋₆-alkyl,
(i) C₁₋₄-alkoxy-C₂₋₄-alkyl,
(j) C₁₋₄-alkylamino-C₂₋₄-alkyl,
(k) di(C₁₋₄-alkyl)amino-C₂₋₄-alkyl,
(l) aminocarbonyl-C₁₋₄-alkyl,
(m) C₂₋₃-acylamino-C₂₋₄-alkyl,
(n) C₁₋₄-alkylthio-C₂₋₄-alkyl,
(o) C₂₋₄-acyl-C₁₋₄-alkyl, and
(p) C₁₋₄-alkylsulfonyl-C₁₋₄-alkyl, or
   two R⁵ groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁵ groups form a piperazine ring, the nitrogen of the piperazine ring that allows the substitution is optionally substituted with C₁₋₄-alkyl;
   R⁶ is independently selected from:
(a) hydrogen,
(b) C₁₋₄-alkyl, and
(c) hydroxy-C₂₋₄-alkyl;
   R⁷ is independently selected from:
(a) hydrogen, and
(b) C₁₋₄-alkyl;
   R⁸ is independently selected from:
(a) hydrogen,
(b) C₁₋₄-alkyl,
(c) CF₃,
(d) C₃₋₅-cycloalkyl,
(e) methyl-C₃₋₅-cycloalkyl, and
(f) C-heterocyclyl, optionally substituted with methyl;
   R⁹ is each independently selected from:
(a) C₁₋₄-alkoxy-C₂₋₄-alkyl,
(b) amino-C₂₋₄-alkyl,
(c) C₁₋₄-alkylamino-C₂₋₄-alkyl,
(d) di(C₁₋₄-alkyl)amino-C₂₋₄-alkyl,
(e) C₂₋₃-acylamino-C₂₋₄-alkyl,
(f) C₁₋₄-alkylthio-C₂₋₄-alkyl, and
(g) C₂₋₄-acyl-C₁₋₄-alkyl, or
   two R⁹ groups together with the nitrogen to which they are attached form a heterocyclic ring, wherein said heterocyclic ring may be optionally substituted with:
   i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁹ groups form a piperazine ring, the nitrogen of the piperazine ring that allows the substitution is optionally substituted with C₁₋₄-alkyl;
   R¹⁰ is independently selected from:
(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) C₁₋₂-alkoxy-C₂₋₃-alkyl,
(h) hydroxy-C₂₋₄-alkyl,
(i) C₂₋₃-acyl,
(j) amino-C₂₋₄-alkyl,
(k) methylamino-C₂₋₄-alkyl,
(l) dimethylamino-C₂₋₄-alkyl,
(m) cyano-C₁₋₄-alkyl, and
(n) tetrahydrofuran-2-ylmethyl;
   one of R¹¹ is hydrogen, and the other R¹¹ is independently selected from:
(a) hydrogen,
(b) halogen selected from chlorine and fluorine,
(c) —S(O)₂CH₃,
(d) —S(O)₂CF₃,
(e) —OS(O)₂CF₃,
(f) —S(O)NH₂,
(g) —S(O)₂NHCH₃,
(h) —S(O)₂N(CH₃)₂,
(i) —NHS(O)₂CH₃,
(j) —N(CH₃)S(O)₂CH₃,
(k) —NHC(O)CH₃,
(l) —N(CH₃)C(O)CH₃,
(m) —C(O)NH₂,
(n) —C(O)NHCH₃,
(o) —C(O)N(CH₃)₂,
(p) —CN,
(q) —CF₃,
(r) guanidino,
(s) amidino,
(t) —OH,
(u) C₁₋₄-alkoxy,
(v) —OCF₃,
(w) C₃₋₅-cycloalkyloxy,
(x) —SCF₃,
(y) —NO₂,
(z) —NR⁵R⁵, wherein each R⁵ is independently selected from the group consisting of hydrogen and C₁₋₄-alkyl; or two R⁵ groups together with the nitrogen to which they are attached form a pyrrolidine or an azetidine ring,
(aa) —C(OH)CH₃CF₃,
(bb) C₁₋₃-alkyl,
(cc) C₁₋₃-alkoxy-C₁₋₂-alkyl,
(dd) C₂₋₃-acyl,
(ee) C₂₋₃-alkenyl,
(ff) hydroxy-C₁₋₄-alkyl,
(gg) fluoro-C₂₋₃-alkyl,
(hh) C₂₋₃-alkynyl, and
(ii) C₃₋₅-cycloalkyl.

A preferred subgroup of compounds of the Formula (I) consists of compounds wherein:

A¹ is CH₂ and B¹ is O or NR¹⁰, or

A¹ is O or NR¹⁰ and B¹ is CH₂;

Ar¹ is phenyl, which is optionally substituted in one or two positions with a substituent independently selected from the group Z⁴ consisting of:

(a) halogen selected from chlorine and fluorine,
(b) C₁₋₄-alkylsulfonyl,
(c) C₁₋₄-alkylsulfinyl,
(d) hydroxy-C₂₋₄-alkylsulfonyl,
(e) C₃₋₅-cycloalkylsulfonyl,
(f) methyl-C₃₋₅-cycloalkylsulfonyl,
(g) trifluoromethylsulfonyl,
(h) —S(O)₂NR^{5A}R^{5A},
(i) C₁₋₄-alkylsulfonamido,
(j) C₂₋₄-acylamino,
(k) C₂₋₄-acylaminomethyl,
(l) carboxy-C₁₋₃-alkylcarbonylamino,
(m) —C(O)NR^{5A}R^{5A},
(n) —CH₂—C(O)NR^{5A}R^{5A}
(o) —NHC(O)OCH₃,
(p) C₂₋₄-acyl,
(q) C₃₋₅-cycloalkylcarbonyl,
(r) C₁₋₄-alkoxy,
(s) C₃₋₅-cycloalkyloxy,
(t) C-heterocyclyl,
(u) —CN,
(v) —OH,
(w) —OCF₃,
(x) —CF₃,
(y) —NO₂,
(z) —NR^{5A}R^{5A},
(aa) —C(OH)CH₃CF₃,
(bb) cyano-C₁₋₂-alkyl,
(cc) C₁₋₄-alkyl,
(dd) C₃₋₅-cycloalkyl,
(ee) C₁₋₂-alkoxy-C₁₋₂-alkyl,
(ff) vinyl,
(gg) ethynyl,
(hh) hydroxy-C₁₋₂-alkyl,
(ii) C-heterocyclyloxy, optionally substituted with methyl,
(jj) R^{5A}R^{5A}N—C₁₋₂-alkyl, and
(kk) —C(O)OR^{7A};
   R₁ is a group R^{1A} selected from C(O)OR^{2A}, C(O)R^{2A}, S(O)₂R^{2A}, C(O)NR^{2A}R^{3A}, and —CH₂—C(O)NR^{2A}R^{3A};
   R^{2A} is selected from:
(a) C₁₋₆-alkyl,
(b) C₁₋₆-alkoxy-C₂₋₆-alkyl,
(c) hydroxy-C₂₋₆-alkyl,
(d) hydroxy-C₂₋₄-alkoxy-C₂₋₄-alkyl,
(e) fluoro-C₂₋₆-alkyl,
(f) C₃₋₆-alkynyl,
(g) C₃₋₇-cycloalkyl,
(h) C₅₋₈-cycloalkenyl,
(i) NR^{9A}R^{9A} provided that R^{1A} is not selected from C(O)OR^{2A}, C(O)NR^{2A}R^{3A} and —CH₂—C(O)NR_{2A}R^{3A},
(j) C-heterocyclyl, optionally substituted with methyl,
(k) C₇₋₈-bicyclyl,
(l) 2-norbornylmethyl,
(m) azabicyclyl,
(n) C₃₋₆-cycloalkyl-C₁₋₄-alkyl, wherein cycloalkyl is optionally substituted with methyl,
(o) C₂₋₃-acyl-C₁₋₄-alkyl,
(p) arylcarbonyl-C₁₋₄-alkyl,
(q) heteroarylcarbonyl-C₄-alkyl,
(r) [C(OH)CH₃CF₃]—C₁₋₆-alkyl,
(s) N-heterocyclylcarbonyl-C₂₋₄-alkyl, wherein heterocyclyl is optionally substituted with methyl,
(t) hydroxy-C₄₋₆-cycloalkyl,
(u) oxo-C₄₋₆-cycloalkyl,
(v) fluoro-C₄₋₆-cycloalkyl,
(w) methoxy-C₄₋₆-cycloalkyl,
(x) methyl-C₃₋₆-cycloalkyl,
(y) oxo-N-heterocyclyl-C₂₋₄-alkyl,
(z) hydroxy-N-heterocyclyl-C₂₋₄-alkyl,
(aa) fluoro-N-heterocyclyl-C₂₋₄-alkyl,
(bb) amino-N-heterocyclyl-C₂₋₄-alkyl,
(cc) N-heterocyclyl-C₂₋₄-alkyl, wherein heterocyclyl is optionally substituted with methyl,
(dd) C-heterocyclyl-C₁₋₄-alkyl, wherein heterocyclyl is optionally substituted with methyl,
(ee) aryl,
(ff) aryl-C₁₋₄-alkyl,
(gg) heteroaryl, and
(hh) heteroaryl-C₁₋₄-alkyl,
   wherein any aryl or heteroaryl residue, alone or as apart of another group, is optionally independently substituted in one or more positions with a substituent selected from the group Z⁵ consisting of:
(a) halogen selected from chlorine and fluorine,
(b) methyl,
(c) ethyl,
(d) methoxy,
(e) ethoxy,
(f) isopropoxy,
(g) hydroxy,
(h) —OCF₃,
(i) —CF₃,
(j) —CN,
(k) —C(OH)CH₃CF₃,
(l) dimethylamino,
(m) hydroxymethyl,
(n) —S(O)₂CH₃,
(o) —C(O)CH₃, and
(p) —C(O)NH₂;
   R^{3A} is selected from:
(a) hydrogen,
(b) C₁₋₄-alkyl,
(c) hydroxy-C₂₋₄-alkyl, and
(d) methoxy-C₂₋₄-alkyl;
   R^{5A} is each independently selected from:
(a) hydrogen,
(b) C₁₋₃-alkyl,
(c) C₁₋₂-alkoxy-C₂₋₄-alkyl,
(d) C₃₋₄-cycloalkyl,
(e) hydroxy-C₂₋₄-alkyl,
(f) cyano-C₁₋₃-alkyl,
(g) dihydroxy-C₂₋₄-alkyl,
(h) aminocarbonyl-C₁₋₂-alkyl, and
(i) di(C₁₋₂-alkyl)amino-C₂₋₃-alkyl,
   or two R^{5A} groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
   ii) one or two oxo groups; or
   iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R^{5A} groups form a piperazine ring, the nitrogen of the piperazine ring that allows the substitution is optionally substituted with methyl;
   R^{7A} is independently from:
(a) hydrogen, and
(b) C₁₋₄-alkyl;
   R^{9A} is each taken together with the nitrogen to which they are attached to form a heterocyclic ring, wherein said heterocyclic ring may be optionally substituted with: i) one hydroxy or amino group, ii) one or two fluorine atoms, or iii) one or two oxo groups, provided that when the substituent is selected from fluorine, hydroxy and amino, said substituent is attached to the heterocyclic ring at a position other than alpha to a heteroatom; and when the two R^{9A} groups form a piperazine ring, the nitrogen of the piperazine ring that allows the substitution is optionally substituted with methyl;
   R¹⁰ is independently selected from:
(a) hydrogen, and
(b) C₁₋₃-alkyl;
   R¹¹ are both hydrogen.

In a more preferred subgroup of compounds of the invention, A¹ is CH₂ and B¹ is NR¹⁰, wherein R¹⁰ is independently selected from hydrogen, methyl and ethyl.

In another more preferred subgroup of compounds of the invention, A¹ is O and B¹ is CH₂.

In another more preferred subgroup of compounds of the invention, Ar¹ is C₁₋₄-alkyl-sulfonylphenyl. Preferably, Ar¹ is methylsulfonylphenyl.

In yet another more preferred subgroup of compounds of the invention, R¹ is C(O)OR². R² is preferably C₁₋₄-alkyl, more preferably tert-butyl.

An even more preferred subgroup of compounds of Formula (I) consists of compounds wherein:

A¹ is CH₂ and B¹ is NR¹⁰, or

A¹ is O and B¹ is CH₂;

Ar¹ is methylsulfonylphenyl;

R¹ is C(O)OR², wherein R² is tert-butyl;

R¹⁰ is independently selected from hydrogen, methyl and ethyl;

R¹¹ are both hydrogen.

Particularly preferred compounds of the invention are the compounds selected from the group consisting of:
tert-Butyl 4-[({6-[4-(methylsulfonyl)phenyl]pyridazin-3-yl}oxy)methyl]piperidine-1-carboxylate;
tert-Butyl 4-[({6-[4-(methylsulfonyl)phenyl]pyridazin-3-yl}methyl)amino]-piperidine-1-carboxylate;
tert-Butyl 4-[methyl({6-[4-(methylsulfonyl)phenyl]pyridazin-3-yl}methyl)amino]-piperidine-1-carboxylate; and
tert-Butyl 4-[ethyl({6-[4-(methylsulfonyl)phenyl]pyridazin-3-yl}methyl)amino]-piperidine-1-carboxylate.

All isomeric forms possible (pure enantiomers, diastereomers, tautomers, racemic mixtures and unequal mixtures of two enantiomers) for the compounds delineated are within the scope of the invention. When the compounds described herein contain olefinic double bonds of geometric asymmetry, it is intended to include both trans and cis (E and Z) geometric isomers.

The compounds of the Formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Another object of the present invention is a compound of Formula (I) for use in therapy. The compound can be used in the treatment or prophylaxis of disorders relating to GPR119. Examples of such disorders are Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is a method for the treatment or prophylaxis of disorders related to GPR119, said method comprising administering to a subject (e.g., mammal, human, or animal) in need of such treatment an effective amount of a compound as described above. The GPR119-related disorder is any disorder or symptom wherein GPR119 is involved in the process or presentation of the disorder or the symptom. The GPR119-related disorders include, but are not limited to Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is a method for modulating the GPR119 receptor activity (e.g., agonizing human GPR119), comprising administering to a subject (e.g., mammal, human, or animal) in need thereof an effective amount of a compound as described above or a composition comprising a compound as described above.

Another object of the present invention is the use of a compound as described above in the manufacture of a medicament for use in the treatment or prophylaxis of Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Another object of the present invention is the use of a compound of Formula (I), as described above, in the manufacture of a medicament for use in the treatment or prophylaxis of disorders related to GPR119, said method comprising administering to a subject (e.g., mammal, human, or animal) in need of such treatment an effective amount of a compound as described above. The GPR119-related disorder is any disorder or symptom wherein GPR119 is involved in the process or presentation of the disorder or the symptom.

The GPR119-related disorders include, but are not limited to, Type 1 and Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, endothelial dysfunction.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabelling/assay techniques, blotting/chemiluminescence methods, real-time PCR, and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

The compounds of formula (I) may be administered with other active compounds for the treatment of diabetes and/or obesity, for example insulin and insulin analogs, DPP-IV inhibitors, sulfonyl ureas, biguanides, α2 agonists, glitazones, PPAR-γ agonists, mixed PPAR-α/γ agonists, RXR agonists, α-glucosidase inhibitors, PTP1B inhibitors, 11-β-hydroxy steroid dehydrogenase Type 1 inhibitors, phosphodiesterase inhibitors, glycogen phosphorylase inhibitors, MCH-1 antagonists, CB-1 antagonists (or inverse agonists), amylin antagonists, CCK receptor agonists, β₃-agonists, leptin and leptin mimetics, serotonergic/dopaminergic antiobesity drugs, gastric lipase inhibitors, pancreatic lipase inhibitors, fatty acid oxidation inhibitors, lipid lowering agents and thyromimetics.

It is particularly preferred that the compounds of formula (I) are administered in combination with a DPP-IV inhibitor. The term “DPP-IV inhibitor” means a compound which inhibits, antagonizes or decreases the activity of dipeptidyl peptidase IV (EC 3.4.14.5). The said DPP-IV inhibitor can e.g. be a compound as disclosed in WO 2005/056003; WO 2005/056013; WO 2005/095343; WO 2005/113510; WO 2005/120494; WO 2005/121131; WO 2005/121089; WO 2006/013104; or WO 2006/076231, including references therein.

The processes described below in the example section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of Formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of Formula (I) and other compounds herein are either known or may be prepared in analogy with the preparation of known compounds.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The invention will now be further illustrated by the following non-limiting Examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

¹H Nuclear magnetic resonance (NMR) and ¹³C NMR were recorded on a Bruker Advance DPX 400 spectrometer at 400.1 MHz and 100.6 MHz, respectively. All spectra were recorded using residual solvent or tetramethylsilane (TMS) as internal standard. Low-resolution electrospray ionization mass spectra (LRESIMS) were obtained using an Agilent MSD mass spectrometer or a Waters ZQ mass spectrometer. High-resolution electrospray ionization mass spectra (HRESIMS) were obtained on an Agilent LC/MSD TOF connected to an Agilent 1100 LC-system, Ion Source: ESI, Ion polarity: pos, Data: profile mode, Scan range: 100-1100 Da, MS parameters: Fragmentor 215V, Skimmer 560V och OCT RF (octpole rods) 250 V.; Reference Masses 121.050873 and 922.009798 (Agilent reference Mix); LC: A 15 mM ammonium acetate; B 100 MeCN; flow 400 μL/min isocratic. Flash chromatography was performed on Merck silica gel 60 (230-400 mesh). The compounds were automatically named using ACD 6.0.

Analytical LCMS Data were Obtained with:

System A: Agilent MSD mass spectrometer; Agilent 1100 system; ACE 3 C8 column (50×3.0 mm); Water containing 0.1% TFA and acetonitrile were used as mobile phases at a flow rate of 1 mL/min with gradient times of 3.0 min. (gradient 10-97% acetonitrile).

Preparative HPLC was Performed on Gilson System Equipped with:

System B: XTerra Prep MS C18 5 μm (19×50 mm) column. Water containing 50 mM NH₄HCO₃ (pH=10) and acetonitrile were used as mobile phases at a flow rate of 25 mL/min with gradient times of 6 min; or

System C: ACE C8 5 μm (21.2×50 mm) column. Water containing 0.1% TFA and acetonitrile were used as mobile phases at a flow rate of 25 mL/min with gradient times of 6 min.

### Example A1

### tert-Butyl 4-[({6-[4-(methylsulfonyl)phenyl]pyridazin-3-yl}oxy)methyl]piperidine-1-carboxylate

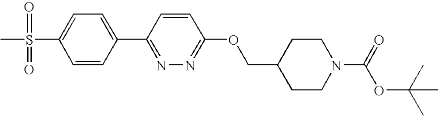

3,6-Dichloropyridazine (0.50 g, 3.36 mmol), tert-butyl 4-(hydroxymethyl)piperidine-1-carboxylate (0.72 g, 3.36 mmol) and potassium tert-butoxide (0.45 g, 4.03 mmol) in dry THF (10 mL) were heated at 60° C. for 48 h. The reaction mixture was filtered and the solvent was removed under reduced pressure. The product, tert-butyl 4-{[(6-chloro-pyridazin-3-yl)oxy]methyl}piperidine-1-carboxylate, was directly used in the next step. tert-Butyl 4-{[(6-chloropyridazin-3-yl)oxy]methyl}piperidine-1-carboxylate (0.24 g, 0.73 mmol) was dissolved in toluene (6 mL) and (4-methylsulfonylphenyl)boronic acid (0.17 g, 0.84 mmol), Na₂CO₃ (0.16 g, 1.55 mmol), Pd(PPh₃)₄ (0.03 g, 0.02 mmol) and water (0.2 mL) were added and the reaction mixture was heated at 110° C. for 16 h. The reaction mixture was filtered and the solvent was removed under reduced pressure. The product was purified by preparative HPLC (System C, using first acetonitrile-water gradients containing 0.1% ammonium acetate and then acetonitrile-water gradients containing 0.1% TFA) to give the title compound. Yield 14.1 mg (4%). Analytical HPLC: purity 95% (System A, R_{T}=2.4 min); ¹H NMR (400 MHz, CD₃OD) δ ppm 1.23-1.35 (m, 2H) 1.44 (s, 9H) 1.82 (s, 2H) 1.98-2.21 (m, 1H) 2.80 (s, 2H) 3.15 (s, 3H) 4.11 (d, J=13.4 Hz, 2H) 4.40 (d, J=6.4 Hz, 2H) 7.28 (m, 1H) 8.05-8.10 (m, 2H) 8.13 (m, 1H) 8.20-8.27 (m, 2H); LRESMS for C₂₂H₂₉N₃O₅S m/z 448 (M+H)⁺.

### Intermediate A1

### 3-Methyl-6-[4-(methylsulfonyl)phenyl]pyridazine

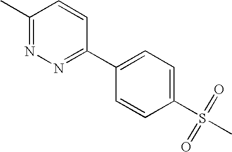

A suspension of 3-chloro-6-methylpyridazine (1.0 g, 7.78 mmol), (4-methylsulfonyl-phenyl)boronic acid (1.71 g, 8.56 mmol), Pd(PPh₃)₄ (450 mg, 0.39 mmol), potassium carbonate (2.69 g, 19.45 mmol) in 1,4-dioxane (25 mL) and water (5 mL) was heated at 95° C. overnight. Upon evaporation of the solvents, the residue was partitioned between water (150 mL) and chloroform (150 mL). The layers were separated and the water phase was extracted with chloroform (2×100 mL). The combined organic layers were evaporated and purified by flash chromatography (1:1 DCM/EtOAc, then 100% EtOAc). Yield 1.36 g (70%); Analytical HPLC: purity 99% (System A, R_{T}=1.04 min); LRESIMS for C₁₂H₁₂N₂O₂S m/z 249 (M+H)⁺.

### Intermediate A2

### 3-(Chloromethyl)-6-[4-(methylsulfonyl)phenyl]pyridazine

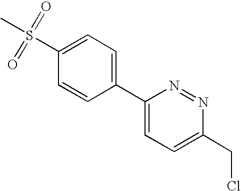

To a reaction tube containing 3-methyl-6-[4-(methylsulfonyl)phenyl]pyridazine (950 mg, 3.826 mmol; Intermediate A1) in chloroform (20 mL) was added trichloroisocyanuric acid (356 mg, 1.530 mmol). The mixture was heated at 80° C. for 20 min, allowed to reach r.t. and then filtered. The filtrate was evaporated and then purified by flash chromatography (gradient DCM→50% EtOAc in DCM). Yield 810 mg (75%); Analytical HPLC: purity 92% (System A, R_{T}=1.53 min); LRESIMS for C₁₂H₁₁ClN₂O₂S m/z 283 (M+H)⁺.

### Example A2

### tert-Butyl 4-[({6-[4-(methylsulfonyl)phenyl]pyridazin-3-yl}methyl)amino]piperidine-1-carboxylate

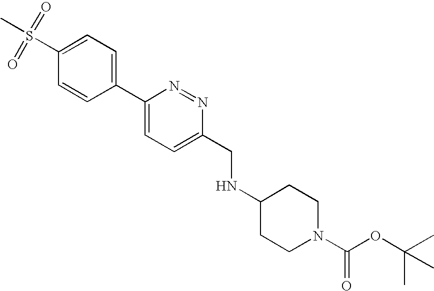

To a flask containing 3-(chloromethyl)-6-[4-(methylsulfonyl)phenyl]pyridazine (400 mg, 1.41 mmol; Intermediate A2) were added tert-butyl-4-aminopiperidine-1-carboxylate (283 mg, 1.41 mmol), N,N-diisopropylethylamine (0.5 mL, 2.82 mmol) and DMF (2 mL). The 25 mixture was stirred at 50° C. for 5 hours and then concentrated under reduced pressure. The residue was purified by preparative HPLC (System B, gradient 12-42% MeCN). Yield 109 mg (17%); Analytical HPLC: purity 100% (System A, R_{T}=1.57 min); ¹H NMR (400 MHz, CDCl₃) δ ppm 1.27-1.42 (m, 2H) 1.47 (s, 9H) 1.88-1.98 (m, 2H) 2.71-2.90 (m, 3H) 3.13 (s, 3H) 3.96-4.14 (m, 2H) 4.25 (s, 2H) 7.73 (m, 1H) 7.92 (m, 1H) 8.09-8.15 (m, 2H) 8.28-8.34 (m, 2H); LRESIMS for C₂₂H₃₀N₄O₄S m/z 447 (M+H)⁺; HRESIMS, calc. monoiso mass (Da): 446.1988. found monoiso mass (Da): 446.1982.

### Example A3

### tert-Butyl 4-[methyl({6-[4-(methylsulfonyl)phenyl]pyridazin-3-yl}methyl)amino]-piperidine-1-carboxylate

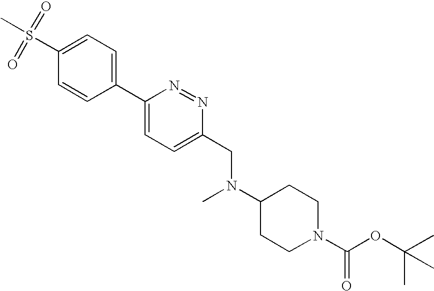

To a reaction tube containing tert-butyl 4-[({6-[4-(methylsulfonyl)phenyl]pyridazin-3-yl}methyl)amino]piperidine-1-carboxylate (25 mg, 0.055 mmol; Example A2), NaBH(OAc)₃ (0.109 mmol) and methanol (1.5 mL) was added a 37% solution of formalin (4 μL, 0.082 mmol). The mixture was stirred overnight and then concentrated under reduced pressure. The residue was purified by preparative HPLC (System B, gradient 20-60% MeCN). Yield 9 mg (36%); Analytical HPLC: purity 100% (System A, R_{T}=1.59 min); ¹H NMR (400 MHz, CDCl₃) δ ppm 1.40 (s, 9H) 1.41-1.51 (m, 2H) 1.74-1.82 (m, 2H) 2.23 (s, 3H) 2.51-2.70 (m, 3H) 3.04 (s, 3H) 3.96 (s, 2H) 4.03-4.19 (m, 2H) 7.71-7.76 (m, 1H) 7.81-7.86 (m, 1H) 8.01-8.06 (m, 2H) 8.20-8.26 (m, 2H); LRESIMS for C₂₃H₃₂N₄O₄S m/z 461 (M+H)⁺; HRESIMS, calc. monoiso mass (Da): 460.2144. found monoiso mass (Da): 460.2149.

### Example A4

### tert-Butyl 4-[ethyl({6-[4-(methylsulfonyl)phenyl]pyridazin-3-yl}methyl)amino]-piperidine-1-carboxylate

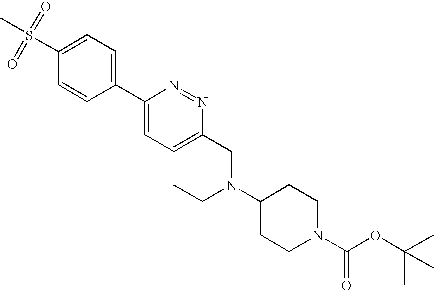

A mixture of tert-butyl 4-[({6-[4-(methylsulfonyl)phenyl]pyridazin-3-yl}methyl)amino]-piperidine-1-carboxylate (15 mg, 0.034 mmol; Example A2), iodoethane (6 μL, 0.068 mmol), potassium carbonate (9 mg, 0.068 mmol) and DMF (1.5 mL) was stirred at r.t. overnight. After evaporation the residue was purified by preparative HPLC (System B, gradient 20-60 MeCN). Yield 1 mg (6%); Analytical HPLC: purity 96% (System A, R_{T}=1.66 min); LRESIMS for C₂₄H₃₄N₄O₄S m/z 475 (M+H)⁺; HRESIMS, calc. monoiso mass (Da): 474.2301. found monoiso mass (Da): 474.2301.

Biological Tests

Human GPR119 Activity Assay

Agonists to the human GPR119 receptor were characterized by measuring human GPR119 receptor-mediated stimulation of cyclic AMP (cAMP) in HEK 293 cells expressing the human GPR119 receptor.

Briefly, cAMP content was determined using a cAMP kit based on HTRF technology (Homogeneous Time-Resolved Fluorescence, Cisbio Cat. no. 62AM2PEC). HEK293 cells stably expressing the human GPR119 receptor (HEK293-hGPR119 cells) were cultured in DMEM (Gibco #31966-021) supplemented with 10% Bovine Calf Serum (Hyclone #SH30072.03), and, 500 μg/mL Hygromycin B (Roche Diagnostics 843555). At 80% confluency, cells were detached using Trypsine and aliquoted at a density of 5×10⁶ cells/mL in freezing medium (DMEM (Gibco # 31966-021), 20% BCS (Hyclone #SH30072.03), 10% DMSO (Sigma #D2650) and stored at −135° C. On the experimental day, HEK293-hGPR119 cells were thawn and diluted to 0.4×10⁶ cells/mL in assay buffer (1×HBSS (Gibco Cat. no. 14025-049), 20 mM Hepes (Gibco Cat. no. 15630-056), 0.1% BSA, pH 7.4) and incubated with test substances for 20 min at room temperature. After addition of HTRF reagents diluted in lysis buffer, the 96- or 384-well plates were incubated 1 hour, followed by measuring the fluorescence ratio at 665 nm/620 nm. Test substances was diluted in compound buffer (1× HBSS (Gibco Cat. no. 14025-049), 20 mM Hepes (Qibco Cat. no. 15630-056), 0.1% BSA, 2 mM IBMx (Sigma-Aldrich Cat. No. 17018, pH 7.4). The potency of the agonist was quantified by determine the concentration that cause 50% activation of hGPR119 evoked increase in cAMP, EC₅₀.

Compounds of the invention showed a concentration-dependant increase in intracellular cAMP level and generally had an EC₅₀ value of <5 μM. Obtained EC₅₀ values for representative compounds of the present invention are shown in Table A.

TABLE A
Agonist potency at the human GPR119.

| Compound | EC₅₀ (μM) |
|---|---|
| A1 | 0.046 |
| A4 | 0.083 |

Effects of GPR119 Modulators on Glucose-Stimulated Insulin Release
In Vitro Experiments

The effect of GPR119 modulators on glucose-stimulated insulin release is determined in isolated pancreatic islets from Wistar rats and diabetic rat models, e.g. GK rat. Briefly, islets are isolated from the rats by digestion with collagenase according to standard protocol. The islets are cultured for 24 h in RPMI-1640 medium supplemented with 11.1 mM glucose and 10% (vol/vol) fetal calf serum. On the experimental day, batches of three islets are preincubated in KRB (Krebs-Ringer bicarbonate) buffer and 3.3 mM glucose for 30 min, 37° C. Thereafter the batches with islets are incubated in 16.7 mM glucose and KRB buffer supplemented with vehicle or test compounds for 60 min at 37° C. Aliquots of the medium will be frozen for measurement of insulin using a radioimmunoassay with rabbit ant-porcine insulin antibodies.

In Vivo Experiments

The effects of GPR119 modulators on glucose stimulated insulin release is determined in diabetic mice models (eg. Lep^{ob/ob} or diet-induced obese (DIO) mice) undergoing an oral 5 glucose tolerance test. Briefly, overnight fasted mice is given either vehicle or test compound at desired doses via oral gavage. Based on the pharmacokinetic of the test compounds, a glucose boluse dose is delivered via oral gavage 30 min-2 hrs following the test compound. Plasma glucose and insulin levels are determined at desired time points over a 2 hour period using blood collection from tail nick. Plasma glucose is determined using a Glucometer and plasma insulin is determined using an insulin ELISA following blood collection in heparinated tubes and centrifugation.

Effects of GPR119 Modulators on GLP-1 Secretion and Body Weight

In Vivo Experiments

The effect of GPR119 modulators on body weight is determined in diabetic and obese mice models, eg. Lep^{ob/ob} or diet-induced obese (DIO) mice. The food intake and body weight gain is measured during subchronic treatment with vehicle or test compound via oral gavage. At the end of the experiment, vena cava blood is collected and e.g. HbA1c, GLP-1, insulin, ALAT, ASAT are measured.

1. A compound of Formula (I) 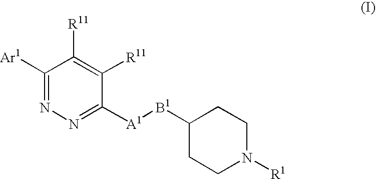
including pharmaceutically acceptable salts, hydrates, geometrical isomers, racemates, tautomers, optical isomers, and N-oxides thereof; wherein:
A¹ is CH₂, O or NR¹⁰;
B¹ is CH₂, O or NR¹⁰, provided that when B¹ is O or NR¹⁰, then A¹ is CH₂;
R¹ is C(O)OR², C(O)R², S(O)₂R², C(O)NR²R³ or —CH₂—C(O)NR²R³;
Ar¹ is phenyl, which is optionally substituted in one or two positions with a substituent independently selected from the group Z¹ consisting of:
(a) CF₃SO₃,
(b) halogen selected from bromine, chlorine and fluorine,
(c) C₁₋₄-alkylsulfinyl,
(d) —S(O)₂R⁴,
(e) —S(O)₂NR⁵R⁵,
(f) —NR⁶S(O)₂R⁴,
(g) —NR⁶C(O)R⁴,
(h) —CH₂—NR⁶C(O)R⁴,
(i) —C(O)NR⁵R⁵,
(j) —CH₂—C(O)NR⁵R⁵,
(k) —C(O)R⁴,
(l) H₂N—C(O)O—,
(m) CH₃—NH—C(O)O—,
(n) (CH₃)₂NC(O)O—,
(o) —NHC(O)OCH₃,
(p) C-heterocyclyl, optionally substituted with methyl,
(q) —CN,
(r) —OR⁸,
(s) —SCF₃,
(t) —NO₂,
(u) phosphonooxy,
(v) C-heterocyclylsulfonyl, optionally substituted with methyl,
(w) —NR⁵R⁵,
(x) —C(OH)CH₃CF₃,
(y) cyano-C₁₋₆-alkyl,
(z) guanidino,
(aa) amidino,
(bb) C₁₋₆-alkyl,
(cc) C₁₋₄-alkoxy-C₁₋₄-alkyl,
(dd) fluoro-C₁₋₄-alkyl,
(ee) C₂₋₆-alkenyl,
(ff) fluoro-C₂₋₄-alkenyl,
(gg) hydroxy-C₁₋₆-alkyl,
(hh) C₁₋₄-alkylsulfonyl-C₁₋₄-alkyl,
(ii) hydroxy-C₂₋₄-alkoxy-C₁₋₄-alkyl,
(jj) C₂₋₃-acyl-C₁₋₃-alkyl,
(kk) C₂₋₆-alkynyl,
(ll) C₃₋₆-cycloalkyl,
(mm) hydroxy-C₃₋₆-cycloalkyl,
(nn) fluoro-C₃₋₆-cycloalkyl,
(oo) methyl-C₃₋₆-cycloalkyl,
(pp) C-heterocyclylcarbonyl, optionally substituted with methyl,
(qq) C₃₋₆-cycloalkyl-C₁₋₄-alkyl,
(rr) R⁵R⁵N—C₁₋₂-alkyl,
(ss) —C(O)OR⁷,
(tt) aryl, and
(uu) heteroaryl,
wherein any aryl or heteroaryl residue as substituent on Ar¹ is optionally independently substituted in one or more positions with a substituent selected from the group Z² consisting of:
(a) halogen selected from chlorine and fluorine,
(b) C₁₋₄-alkyl,
(c) hydroxy,
(d) C₁₋₄-alkoxy,
(e) —OCF₃,
(f) —SCF₃,
(g) —CN,
(h) —C(OH)CH₃CF₃,
(i) hydroxy-C₁₋₄-alkyl,
(j) —CF₃,
(k) —S(O)₂CH₃,
(l) —S(O)₂NH₂,
(m) —S(O)₂NHCH₃,
(n) —S(O)₂N(CH₃)₂,
(o) —N(CH₃)S(O)₂CH₃,
(p) —N(CH₃)C(O)CH₃,
(q) —C(O)NH₂,
(r) —C(O)NHCH₃,
(s) —C(O)N(CH₃)₂,
(t) —C(O)CH₃,
(u) —NH₂,
(v) —NHCH₃,
(w) —N(CH₃)₂,
(x) —NO₂, and
(y) methoxycarbonyl;
R² is selected from:
(a) C₁₋₆-alkyl,
(b) C₁₋₆-alkoxy-C₂₋₆-alkyl,
(c) hydroxy-C₂₋₆-alkyl,
(d) fluoro-C₂₋₆-alkyl,
(e) C₃₋₆-alkynyl,
(f) C₃₋₆-alkenyl,
(g) C₃₋₇-cycloalkyl,
(h) C₅₋₈-cycloalkenyl,
(i) NR⁹R⁹, provided that R¹ is not selected from C(O)OR², C(O)NR²R³ and —CH₂—C(O)NR²R³,
(j) C-heterocyclyl, optionally substituted with C₁₋₄-alkyl,
(k) C₇₋₈-bicyclyl, optionally substituted with hydroxy,
(l) C₇₋₈-bicyclylmethyl,
(m) azabicyclyl, optionally substituted with hydroxy,
(n) C₃₋₇-cycloalkyl-C₁₋₄-alkyl, wherein cycloalkyl is optionally substituted with methyl,
(o) C₁₋₆-alkylsulfonyl-C₂₋₆-alkyl,
(p) C₂₋₃-acyl-C₁₋₄-alkyl,
(q) arylcarbonyl-C₁₋₄-alkyl,
(r) heteroarylcarbonyl-C₁₋₄-alkyl,
(s) [C(OH)CH₃CF₃]-C₁₋₆-alkyl,
(t) N-heterocyclylcarbonyl-C₂₋₄-alkyl, wherein heterocyclyl is optionally substituted with methyl,
(u) C-heterocyclylcarbonyl-C₂₋₄-alkyl, wherein heterocyclyl is optionally substituted with methyl,
(v) aminocarbonyl-C₂₋₆-alkyl,
(w) C₁₋₃-alkylaminocarbonyl-C₂₋₆-alkyl,
(x) di(C₁₋₃-alkyl)aminocarbonyl-C₂₋₆-alkyl,
(y) hydroxy-C₂₋₄-alkoxy-C₂₋₄-alkyl,
(z) hydroxy-C₄₋₆-cycloalkyl,
(aa) oxo-C₄₋₆-cycloalkyl,
(bb) fluoro-C₄₋₆-cycloalkyl,
(cc) C₁₋₃-alkoxy-C₄₋₆-cycloalkyl,
(dd) methyl-C₃₋₆-cycloalkyl,
(ee) oxo-N-heterocyclyl-C₂₋₄-alkyl,
(ff) fluoro-N-heterocyclyl-C₂₋₄-alkyl,
(gg) amino-N-heterocyclyl-C₂₋₄-alkyl,
(hh) hydroxy-N-heterocyclyl-C₂₋₄-alkyl,
(ii) N-heterocyclyl-C₂₋₄-alkyl, wherein heterocyclyl is optionally substituted with methyl,
(jj) C-heterocyclyl-C₁₋₄-alkyl, wherein heterocyclyl is optionally substituted with methyl,
(kk) aryl,
(ll) aryl-C₁₋₄-alkyl,
(mm) aryl-C₃₋₆-alkenyl,
(nn) aryl-C₃₋₆-alkynyl,
(oo) heteroaryl,
(pp) heteroaryl-C₁₋₄-alkyl,
(qq) heteroaryl-C₃₋₆-alkenyl, and
(rr) heteroaryl-C₃₋₆-alkynyl,
wherein any aryl or heteroaryl residue, alone or as part of another group, is optionally independently substituted in one or more position with a substituent selected from the group Z² as defined above;
R³ is selected from:
(a) hydrogen,
(b) C₁₋₆-alkyl,
(c) fluoro-C₂₋₆-alkyl,
(d) hydroxy-C₂₋₆-alkyl,
(e) C₁₋₆-alkoxy-C₂₋₆-alkyl,
(f) amino-C₂₋₆-alkyl,
(g) C₁₋₃-alkylamino-C₂₋₆-alkyl,
(h) di(C₁₋₃-alkyl)amino-C₂₋₆-alkyl,
(i) cyano-C₁₋₆-alkyl, and
(j) C₁₋₆-alkylsulfonyl-C₂₋₆-alkyl;
R⁴ is independently selected from:
(a) C₁₋₆-alkyl,
(b) fluoro-C₁₋₆-alkyl,
(c) hydroxy-C₂₋₆-alkyl,
(d) C₁₋₄-alkoxy-C₂₋₄-alkyl,
(e) C₂₋₄-acyl-C₁₋₄-alkyl,
(f) carboxy-C₁₋₃-alkyl,
(g) C₃₋₆-cycloalkyl,
(h) oxo-C₄₋₆-cycloalkyl,
(i) hydroxy-C₄₋₆-cycloalkyl,
(j) fluoro-C₄₋₆-cycloalkyl,
(k) methyl-C₃₋₆-cycloalkyl,
(l) N-heterocyclylcarbonyl-C₂₋₄-alkyl, wherein heterocyclyl is optionally substituted with methyl,
(m) oxo-N-heterocyclyl-C₂₋₄-alkyl,
(n) fluoro-N-heterocyclyl-C₂₋₄-alkyl,
(o) hydroxy-N-heterocyclyl-C₂₋₄-alkyl,
(p) amino-N-heterocyclyl-C₂₋₄-alkyl,
(q) aminocarbonyl-C₂₋₄-alkyl,
(r) C₁₋₃-alkylaminocarbonyl-C₂₋₄-alkyl,
(s) di(C₁₋₃-alkyl)aminocarbonyl-C₂₋₄-alkyl,
(t) C₂₋₃-acylamino-C₂₋₄-alkyl,
(u) hydroxy-C₂₋₄-alkoxy-C₂₋₄-alkyl,
(v) C-heterocyclylcarbonyl-C₂₋₄-alkyl, wherein heterocyclyl is optionally substituted with methyl,
(w) C₃₋₆-cycloalkyl-C₁₋₂-alkyl,
(x) aryl,
(y) aryl-C₁₋₂-alkyl,
(z) heteroaryl, and
(aa) heteroaryl-C₁₋₂-alkyl,
wherein any aryl or heteroaryl residue, alone or as part of another group, is optionally independently substituted in one or more positions with a substituent selected from the group Z³ consisting of:
(a) halogen selected from chlorine and fluorine,
(b) C₁₋₄-alkoxy,
(c) hydroxymethyl,
(d) —CN,
(e) —CF₃,
(f) C₁₋₄-alkyl,
(g) —OCF₃, and
(h) —C(O)CH₃;
R⁵ is each independently selected from:
(a) hydrogen,
(b) C₁₋₆-alkyl,
(c) C₃₋₄-cycloalkyl,
(d) fluoro-C₂₋₄-alkyl,
(e) amino-C₂₋₆-alkyl,
(f) cyano-C₁₋₆-alkyl,
(g) hydroxy-C₂₋₆-alkyl,
(h) dihydroxy-C₂₋₆-alkyl,
(i) C₁₋₄-alkoxy-C₂₋₄-alkyl,
(j) C₁₋₄-alkylamino-C₂₋₄-alkyl,
(k) di(C₁₋₄-alkyl)amino-C₂₋₄-alkyl,
(l) aminocarbonyl-C₁₋₄-alkyl,
(m) C₂₋₃-acylamino-C₂₋₄-alkyl,
(n) C₁₋₄-alkylthio-C₂₋₄-alkyl,
(o) C₂₋₄-acyl-C₁₋₄-alkyl, and
(p) C₁₋₄-alkylsulfonyl-C₁₋₄-alkyl, or
two R⁵ groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁵ groups form a piperazine ring, the nitrogen of the piperazine ring that allows the substitution is optionally substituted with C₁₋₄-alkyl;
R⁶ is independently selected from:
(a) hydrogen,
(b) C₁₋₄-alkyl, and
(c) hydroxy-C₂₋₄-alkyl;
R⁷ is independently selected from:
(a) hydrogen, and
(b) C₁₋₄-alkyl;
R⁸ is independently selected from:
(a) hydrogen,
(b) C₁₋₄-alkyl,
(c) CF₃,
(d) C₃₋₅-cycloalkyl,
(e) methyl-C₃₋₅-cycloalkyl, and
(f) C-heterocyclyl, optionally substituted with methyl;
R⁹ is each independently selected from:
(a) C₁₋₄-alkoxy-C₂₋₄-alkyl,
(b) amino-C₂₋₄-alkyl,
(c) C₁₋₄-alkylamino-C₂₋₄-alkyl,
(d) di(C₁₋₄-alkyl)amino-C₂₋₄-alkyl,
(e) C₂₋₃-acylamino-C₂₋₄-alkyl,
(f) C₁₋₄-alkylthio-C₂₋₄-alkyl, and
(g) C₂₋₄-acyl-C₁₋₄-alkyl, or
two R⁹ groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R⁹ groups form a piperazine ring, the nitrogen of the piperazine ring that allows the substitution is optionally substituted with C₁₋₄-alkyl;
R¹⁰ is independently selected from:
(a) hydrogen,
(b) C₁₋₄-alkyl,
(c) cyclopropyl,
(d) cyclobutyl,
(e) cyclopropylmethyl,
(f) fluoro-C₂₋₄-alkyl,
(g) C₁₋₂-alkoxy-C₂₋₃-alkyl,
(h) hydroxy-C₂₋₄-alkyl,
(i) C₂₋₃-acyl,
(j) amino-C₂₋₄-alkyl,
(k) methylamino-C₂₋₄-alkyl,
(l) dimethylamino-C₂₋₄-alkyl,
(m) cyano-C₁₋₄-alkyl, and
(n) tetrahydrofuran-2-ylmethyl;
one of R¹¹ is hydrogen, and the other R¹¹ is selected from:
(a) hydrogen,
(b) halogen selected from chlorine and fluorine,
(c) —S(O)₂CH₃,
(d) —S(O)₂CF₃,
(e) —OS(O)₂CF₃,
(f) —S(O)NH₂,
(g) —S(O)₂NHCH₃,
(h) —S(O)₂N(CH₃)₂,
(i) —NHS(O)₂CH₃,
(j) —N(CH₃)S(O)₂CH₃,
(k) —NHC(O)CH₃,
(l) —N(CH₃)C(O)CH₃,
(m) —C(O)NH₂,
(n) —C(O)NHCH₃,
(o) —C(O)N(CH₃)₂,
(p) —CN,
(q) —CF₃,
(r) guanidino,
(s) amidino,
(t) —OH,
(u) C₁₋₄-alkoxy,
(v) —OCF₃,
(w) C₃₋₅-cycloalkyloxy,
(x) —SCF₃,
(y) —NO₂,
(z) —NR⁵R⁵, wherein each R⁵ is independently selected from the group consisting of hydrogen and C₁₋₄-alkyl; or two R⁵ groups together with the nitrogen to which they are attached form a pyrrolidine or an azetidine ring,
(aa) —C(OH)CH₃CF₃,
(bb) C₁₋₃-alkyl,
(cc) C₁₋₃-alkoxy-C₁₋₂-alkyl,
(dd) C₂₋₃-acyl,
(ee) C₂₋₃-alkenyl,
(ff) hydroxy-C₁₋₄-alkyl,
(gg) fluoro-C₂₋₃-alkyl,
(hh) C₂₋₃-alkynyl, and
(ii) C₃₋₅-cycloalkyl.

2. A compound according to claim 1, wherein
A¹ is CH₂ and B¹ is O or NR¹⁰, or
A¹ is O or NR¹⁰ and B¹ is CH₂.

3. A compound according to claim 2, wherein
Ar¹ is phenyl, which is optionally substituted in one or two positions with a substituent independently selected from the group Z⁴ consisting of:
(a) halogen selected from chlorine and fluorine,
(b) C₁₋₄-alkylsulfonyl,
(c) C₁₋₄-alkylsulfinyl,
(d) hydroxy-C₂₋₄-alkylsulfonyl,
(e) C₃₋₅-cycloalkylsulfonyl,
(f) methyl-C₃₋₅-cycloalkylsulfonyl,
(g) trifluoromethylsulfonyl,
(h) —S(O)₂NR^{5A}R^{5A},
(i) C₁₋₄-alkylsulfonamido,
(j) C₂₋₄-acylamino,
(k) C₂₋₄-acylaminomethyl,
(l) carboxy-C₁₋₃-alkylcarbonylamino,
(m) —C(O)NR^{5A}R^{5A},
(n) —CH₂—C(O)NRA R^{5A}
(o) —NHC(O)OCH₃,
(p) C₂₋₄-acyl,
(q) C₃₋₅-cycloalkylcarbonyl,
(r) C₁₋₄-alkoxy,
(s) C₃₋₅-cycloalkyloxy,
(t) C-heterocyclyl,
(u) —CN,
(v) —OH,
(w) —OCF₃,
(x) —CF₃,
(y) —NO₂,
(z) —NR^{5A}R^{5A},
(aa) —C(OH)CH₃CF₃,
(bb) cyano-C₁₋₂-alkyl,
(cc) C₁₋₄-alkyl,
(dd) C₃₋₅-cycloalkyl,
(ee) C₁₋₂-alkoxy-C₁₋₂-alkyl,
(ff) vinyl,
(gg) ethynyl,
(hh) hydroxy-C₁₋₂-alkyl,
(ii) C-heterocyclyloxy, optionally substituted with methyl,
(jj) R^{5A}R^{5A}N—C₁₋₂-alkyl, and
(kk) —C(O)OR^{7A};
R¹ is a group R^{1A} selected from C(O)OR^{2A}, C(O)R^{2A}, S(O)₂R^{2A}, C(O)NR^{2A}R^{3A}, and —CH₂—C(O)NR^{2A}R^{3A};
R^{2A} is selected from:
(a) C₁₋₆-alkyl,
(b) C₁₋₆-alkoxy-C₂₋₆-alkyl,
(c) hydroxy-C₂₋₆-alkyl,
(d) hydroxy-C₂₋₄-alkoxy-C₂₋₄-alkyl,
(e) fluoro-C₂₋₆-alkyl,
(f) C₃₋₆-alkynyl,
(g) C₃₋₇-cycloalkyl,
(h) C₅₋₈-cycloalkenyl,
(i) NR^{9A}R^{9A} provided that R^{1A} is not selected from C(O)OR^{2A}, C(O)NR^{2A}R^{3A} and —CH₂—C(O)NR^{2A}R^{3A},
(j) C-heterocyclyl, optionally substituted with methyl,
(k) C₇₋₈-bicyclyl,
(l) 2-norbornylmethyl,
(m) azabicyclyl,
(n) C₃₋₆-cycloalkyl-C₁₋₄-alkyl, wherein cycloalkyl is optionally substituted with methyl,
(o) C₂₋₃-acyl-C₁₋₄-alkyl,
(p) arylcarbonyl-C₁₋₄-alkyl,
(q) heteroarylcarbonyl-C₁₋₄-alkyl,
(r) [C(OH)CH₃CF₃]-C₁₋₆-alkyl,
(s) N-heterocyclylcarbonyl-C₂₋₄-alkyl, wherein heterocyclyl is optionally substituted with methyl,
(t) hydroxy-C₄₋₆-cycloalkyl,
(u) oxo-C₄₋₆-cycloalkyl,
(v) fluoro-C₄₋₆-cycloalkyl,
(w) methoxy-C₄₋₆-cycloalkyl,
(x) methyl-C₃₋₆-cycloalkyl,
(y) oxo-N-heterocyclyl-C₂₋₄-alkyl,
(z) hydroxy-N-heterocyclyl-C₂₋₄-alkyl,
(aa) fluoro-N-heterocyclyl-C₂₋₄-alkyl,
(bb) amino-N-heterocyclyl-C₂₋₄-alkyl,
(cc) N-heterocyclyl-C₂₋₄-alkyl, wherein heterocyclyl is optionally substituted with methyl,
(dd) C-heterocyclyl-C₁₋₄-alkyl, wherein heterocyclyl is optionally substituted with methyl,
(ee) aryl,
(ff) aryl-C₁₋₄-alkyl,
(gg) heteroaryl, and
(hh) heteroaryl-C₁₋₄-alkyl,
wherein any aryl or heteroaryl residue, alone or as apart of another group, is optionally independently substituted in one or more positions with a substituent selected from the group Z⁵ consisting of:
(a) halogen selected from chlorine and fluorine,
(b) methyl,
(c) ethyl,
(d) methoxy,
(e) ethoxy,
(f) isopropoxy,
(g) hydroxy,
(h) —OCF₃,
(i) —CF₃,
(j) —CN,
(k) —C(OH)CH₃CF₃,
(l) dimethylamino,
(m) hydroxymethyl,
(n) —S(O)₂CH₃,
(o) —C(O)CH₃, and
(p) —C(O)NH₂;
R^{3A} is selected from:
(a) hydrogen,
(b) C₁₋₄-alkyl,
(c) hydroxy-C₂₋₄-alkyl, and
(d) methoxy-C₂₋₄-alkyl;
R^{5A} is each independently selected from:
(a) hydrogen,
(b) C₁₋₃-alkyl,
(c) C₁₋₂-alkoxy-C₂₋₄-alkyl,
(d) C₃₋₄-cycloalkyl,
(e) hydroxy-C₂₋₄-alkyl,
(f) cyano-C₁₋₃-alkyl,
(g) dihydroxy-C₂₋₄-alkyl,
(h) aminocarbonyl-C₁₋₂-alkyl, and
(i) di(C₁₋₂-alkyl)amino-C₂₋₃-alkyl, or
two R^{5A} groups together with the nitrogen to which they are attached form a heterocyclic ring, wherein said heterocyclic ring may be optionally substituted with:
i) a substituent selected from:
(aa) hydroxy,
(bb) amino,
(cc) methylamino,
(dd) dimethylamino,
(ee) hydroxymethyl, and
(ff) aminomethyl;
ii) one or two oxo groups; or
iii) one or two fluorine atoms, provided that when the substituent is selected from fluorine, hydroxy, amino, methylamino and dimethylamino, said substituent is attached to the heterocyclic ring at a position other than alpha to a heteroatom; and when the two R^{5A} groups form a piperazine ring, the nitrogen of the piperazine ring that allows the substitution is optionally substituted with methyl;
R^{7A} is independently from:
(a) hydrogen, and
(b) C₁₋₄-alkyl;
R^{9A} is each taken together with the nitrogen to which they are attached to form a heterocyclic ring, wherein said heterocyclic ring may be optionally substituted with: i) one hydroxy or amino group, ii) one or two fluorine atoms, or iii) one or two oxo groups, provided that when the substituent is selected from fluorine, hydroxy and amino, said substituent is attached to the heterocyclic ring at a position other than alpha to a heteroatom; and when the two R^{9A} groups form a piperazine ring, the nitrogen of the piperazine ring that allows the substitution is optionally substituted with methyl;
R¹⁰ is independently selected from:
(a) hydrogen, and
(b) C₁₋₃-alkyl;
R¹¹ are both hydrogen.

4. A compound according to any one of claims 1 to 3, wherein R¹¹ are both hydrogen.

5. A compound according to any one of claims 1 to 3, wherein A¹ is CH₂ and B¹ is N¹⁰.

6. A compound according to any one of claims 1 to 3, wherein R¹⁰ is independently selected from hydrogen, methyl and ethyl.

7. A compound according to any one of claims 1 to 3, wherein A¹ is O and B¹ is CH₂.

8. A compound according to any one of claims 1 to 3, wherein R¹ is C(O)OR².

9. A compound according to claim 1, wherein R¹ is C(O)OR² and R² is C₁₋₄-alkyl.

10. A compound according to claim 1, wherein Ar¹ is C₁₋₄-alkylsulfonylphenyl.

11. A compound according to claim 1, which is selected from the group consisting of:
tert-Butyl 4-[({6-[4-(methylsulfonyl)phenyl]pyridazin-3-yl}oxy)methyl]-piperidine-1-carboxylate;
tert-Butyl 4-[({6-[4-(methylsulfonyl)phenyl]pyridazin-3-yl}methyl)amino]-piperidine-1-carboxylate;
tert-Butyl 4-[methyl({6-[4-(methylsulfonyl)phenyl]pyridazin-3-yl}methyl)-amino]piperidine-1-carboxylate; and
tert-Butyl 4-[ethyl({6-[4-(methylsulfonyl)phenyl]pyridazin-3-yl}methyl)-amino]piperidine-1-carboxylate.

12. A method for the treatment or prophylaxis of a disorder relating to GPR119 activity which comprises administering to a mammal, including man, in need of such treatment an effective amount of a compound according to claim 1.

13. The method of claim 12, wherein said disorder relating to GPR119 activity is selected from the group consisting of Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

14. A pharmaceutical formulation containing a compound according to claim 1 as active ingredient in combination with a pharmaceutically acceptable diluent or carrier.

15. A method for the treatment or prophylaxis of a disorder relating to GPR119 activity which comprises administering to a mammal, including man, in need of such treatment an effective amount of a compound according to claim 1 in combination with a DPP-IV inhibitor.

16. The method of claim 15, wherein said disorder relating to GPR119 activity is selected from the group consisting of Type 1 diabetes, Type 2 diabetes, inadequate glucose tolerance, insulin resistance, hyperglycemia, hyperlipidemia, hypercholesterolemia, dyslipidemia, syndrome X, metabolic syndrome, obesity, hypertension, chronic systemic inflammation, retinopathy, neuropathy, nephropathy, atherosclerosis, reduced fibrinolysis, and endothelial dysfunction.

17. The pharmaceutical formulation according to claim 14 which in addition comprises a DPP-IV inhibitor.

18. The compound according to claim 1, wherein R¹¹ are both hydrogen, A¹ is CH₂ and B¹ is NR¹⁰.

19. The compound according to claim 1, wherein R¹¹ are both hydrogen, A¹ is O and B¹ is CH₂.

20. The compound according to claim 18, wherein R¹⁰ is independently selected from hydrogen, methyl and ethyl.

21. The compound according to claim 1, wherein R¹¹ are both hydrogen, Ar¹ is C₁₋₄-alkylsulfonylphenyl, A¹ is CH₂ and B¹ is NR¹⁰, R¹ is C(O)OR² and R² is C₁₋₄-alkyl, and R¹⁰ is independently selected from hydrogen, methyl and ethyl.

22. The compound according to claim 1, wherein R¹¹ are both hydrogen, Ar¹ is C₁₋₄-alkylsulfonylphenyl, A¹ is O and B¹ is CH₂, R¹ is C(O)OR² and R² is C₁₋₄-alkyl.
